(19) United States
(12) Patent Application Publication
(10) Pub. No.: US 2025/0002863 A1
(43) Pub. Date: Jan. 2, 2025

(54) METHOD OF GENERATING FUNCTIONAL ISLETS FROM PLURIPOTENT STEM CELLS

(71) Applicant: HANGZHOU REPROGENIX BIOSCIENCE, INC., Hangzhou, ZhejiangHangzhou, Zhejiang (CN)

(72) Inventors: Hongkui DENG, Hangzhou, Zhejiang (CN); Yuanyuan DU, Hangzhou, Zhejiang (CN); Zhen LIANG, Hangzhou, Zhejiang (CN); Xaiofeng WANG, Hangzhou, Zhejiang (CN)

(21) Appl. No.: 18/714,669

(22) PCT Filed: Nov. 30, 2021
(86) PCT No.: PCT/CN2021/134632

§ 371 Date: May 30, 2024

(51) Int. Cl. C12N 5/071; A61K 35/39; A61P 3/10

(52) U.S. Cl. CPC C12N 5/0678; A61K 35/39; A61P 3/10; C12N 2500/38; C12N 2501/11; C12N 2501/117; C12N 2501/15; C12N 2501/155; C12N 2501/16; C12N 2501/395; C12N 2501/415; C12N 2501/727

(57) ABSTRACT

Provided is a method of in vitro generating functional hPSC-islets, comprising a step of generating pancreatic endocrine progenitors from pancreatic progenitors by using a medium supplemented with ISX9 or a combination of ISX9 and Wnt-C59. Also provided are media used in the method, a population of cells including functional hPSC-islets generated by the method and uses thereof.

### TECHNICAL FIELD

The present disclosure relates to biotechnology, and more particularly, to a method, a combination of agents, and a kit for in vitro generating functional hPSC-islets (human pluripotent stem cell-derived islets). The present disclosure also relates to a population of functional hPSC-islets which is obtainable by the method and comprises C-peptide⁺ cells, glucagon⁺ cells and somatostatin⁺ cells, a pharmaceutical composition comprising the population of functional hPSC-islets, a method for treating a mammal having, or at risk of having, diabetes by administering the functional hPSC-islets.

### BACKGROUND

Cell replacement therapy holds promise in the treatment of diseases such as type 1 diabetes mellitus (T1DM), which is mainly caused by the loss of islet B cells. Human islet transplantation has been shown to reverse T1DM by effectively restoring endogenous insulin secretion in patients. However, the widespread application of islet transplantation is severely hindered by the lack of a readily accessible source of human islets.

CN102899288A discloses a method for differentiation of human islet-derived pancreatic stem cells into insulin-producing cells. The method starts with collecting human islet and expanding pancreatic stem cells therefrom. The starting material is a donor-derived tissue not readily accessible, which limits the use of said method.

Several research groups have reported in vitro methods for obtaining insulin-producing cells from human pluripotent stem cells (hPSCs), in which a near homogenous population of pancreatic fate committed PDX1+progenitor cells could be obtained (A. Rezania. Et al., Nat Biotechnol. 2014 November; 32 (11): 1121-33; F. W. Pagliuca, et al., Cell 159, 428-439, Oct. 9, 2014). However, challenges remained in the subsequent commitment of these progenitors to pancreatic β cells at high efficiency. Moreover, the feasibility of this stem cell-based therapeutic strategy has not been systematically assessed, especially in a large animal model physiologically similar to human such as nonhuman primates. Uncertainties still exist in translating a treatment using pluripotent stem cell-derived islets proven to be successful in laboratory on mouse to clinical use on human, especially to transplantation and therapy that are difficult to investigate using rodent animal models.

Accordingly, there remains needs for an efficient and reproducible method for differentiating functional islet suitable for preclinical or clinical use from a readily accessible source.

### SUMMARY

The present inventors have established a differentiation protocol with high efficiency and good reproducibility, which is a prerequisite to meeting the cell quantity and quality thresholds of preclinical and translational research. The inventors focused on optimizing the differentiation protocol from pancreatic progenitor commitment to β cell fate decision by modulating signaling pathways and reconstructing spatial structure of islets. It has been found that two factors were critical: first, the formation of dense, three-dimensional cell aggregates of posterior foregut-committed cells facilitated the efficient generation of NKX6.1+C-peptide+cells; second, adding the small molecule ISX9 at Stage 5 promoted the terminal differentiation of pancreatic endocrine progenitors, and synergistic effect was achieved when ISX9 was added in combination with Wnt-C59. With this optimized protocol, the inventors were able to efficiently and reproducibly generate relatively uniform, islet-sized aggregates which were proven to be safe and efficient in nonhuman primate model after transplantation, thus completing the invention.

Therefore, in general, the present disclosure provides a method of in vitro generating functional hPSC-islets, agents and compositions used in the method, a population of functional hPSC-islets obtainable by the method, a pharmaceutical composition comprising the population of functional hPSC-islets, a method for treating a mammal having or at risk of having diabetes, and a kit for generating functional hPSC-islets.

In a first aspect, the present disclosure provides a method of in vitro generating functional hPSC-islets which comprises:
(1) culturing the hPSCs in a sixth culture medium to obtain cells expressing markers characteristic of the definitive endoderm;
(2) culturing the cells obtained in step (1) in a fifth culture medium to obtain cells expressing markers characteristic of primitive gut tube cells;
(3) culturing the cells obtained in step (2) in a fourth culture medium to obtain cells expressing markers characteristic of posterior foregut cells;
(4) culturing the cells obtained in step (3) in a third culture medium to obtain cells expressing markers characteristic of pancreatic progenitors;
(5) culturing the cells obtained in step (4) in a second culture medium to obtain cells expressing markers characteristic of pancreatic endocrine progenitors;
(6) culturing the cells obtained in step (5) in a first culture medium to obtain cells expressing markers characteristic of functional hPSC-islets;
wherein the second culture medium is supplemented with ISX9 or Wnt-C59, preferably ISX9.

In a further embodiment, the second culture medium is supplemented with a small molecule combination of ISX9 and Wnt-C59.

According to some embodiments of present disclosure, addition of ISX9 and preferably also Wnt-C59 synergistically promotes the differentiation of pancreatic progenitors into pancreatic endocrine progenitors.

In another embodiment of the first aspect, the first culture medium is supplemented with one or more of an ALK5 inhibitor, an Adenylyl cyclase activator, an Axl inhibitor, an IκB kinase inhibitor, a thyroid hormone and ZnSO₄.

According to some embodiments of present disclosure, the present inventors provide a method of generating functional islets and demonstrates that transplantation of these islets into diabetic animal models, including rodent model and nonhuman primate model, effectively restored endogenous insulin secretion and improved glycemic control.

In some embodiments, after transplantation under the kidney capsule of streptozotocin (STZ)-induced diabetic mice, hPSC-islets survived with marked vascularization and preserved cellular complexity, shown by the presence of C-peptide⁺β cells, GCG⁺α cells and SST⁺δ cells. Fasting blood glucose levels of transplanted mice were restored to physiological levels, accompanied by increase in body weights. Glucose tolerance tests showed glucose-responsive human C-peptide secretion, as well as rapid glucose clearance. Fasting human C-peptide secretion increased steadily from 2 to 12 wpt, after which it was maintained at around 1 ng/ml for up to 36 weeks in non-diabetic mice. Notably, the 15-week survival rate of hPSC-islet transplanted diabetic mice was over 85%, compared to less than 20% in the non-transplanted control group.

In some embodiments, after a one-dose intraportal infusion of stem cell-derived islets in nonhuman primate model, fasting blood glucose and average prepandial blood glucose levels significantly decreased in all recipients. Importantly, three months after transplantation, the average HbA1c dropped by over 2% compared with peak values, while the average exogenous insulin requirement reduced by 49% 15 weeks after transplantation. Furthermore, C-peptide release in response to meals, glucose and arginine was observed in the recipients. Notably, these improvements were also observed in a recipient macaque possessing a glycemic status resembling that in patients with labile diabetes, in whom islet replacement therapy confers significant clinical and potentially life-saving benefits. Collectively, our findings demonstrated the feasibility of pluripotent stem cell-derived islets for diabetic treatment in a pre-clinical context, which marks a significant step forward in the process of clinical translation of hPSC-islets.

In a second aspect, the present disclosure provides a population of functional hPSC-islets obtainable by the method of the first aspect. The population of functional hPSC-islets may be used to treat a mammal having, or at risk of having, type I diabetes, type II diabetes, pre-diabetes or any combination thereof, for example by transplanting these islets in a subject in need of such treatment.

In a third aspect, the present disclosure provides a pharmaceutical composition comprising the population of functional hPSC-islets of the first aspect. The pharmaceutical composition may be used to treat a mammal having, or at risk of having, type I diabetes, type II diabetes, pre-diabetes or any combination thereof, for example by transplanting these islets in a subject in need of such treatment.

In a fourth aspect, the present disclosure provides a method for treating a mammal having, or at risk of having, type I diabetes, type II diabetes, pre-diabetes or any combination thereof, comprising administering to the mammal the population of functional hPSC-islets of the second aspect in a therapeutically effective amount or the pharmaceutical composition of the third aspect.

In a fifth aspect, the present disclosure provides a kit for generating functional hPSC-islets, comprising: at least one of a first to a seventh culture medium.

In a specific embodiment of the fifth aspect, the second culture medium comprises ISX9 or Wnt-C59, preferably ISX9. In a further embodiment of the fifth aspect, the second culture medium comprises a small molecule combination of ISX9 and Wnt-C59.

In a sixth aspect, the present disclosure relates to use of ISX9 alone or a combination of ISX9 and Wnt-C59 in inducing differentiation of pancreatic progenitors into pancreatic endocrine progenitors.

### BRIEF DESCRIPTION OF THE DRAWINGS

The above and other objects, features and advantages will be more apparent from the following description of embodiments with reference to the figures, in which:

FIG. 1 illustrates establishment of an efficient protocol generated functional hPSC-derived islets in vitro that reverse diabetes in diabetic mice in vivo. a, Schematic of the differentiation protocol. b, Left: representative bright field image of Stage 6 cell aggregates. Scale bar, 500 μm. Right: representative flow cytometry analysis of the expression of β cell markers in the cell aggregates at day 3 of Stage 6 (S6D3). c, Representative immunostaining of islet hormones in sectioned Stage 6 aggregate. Scale bar, 50 μm. d, Proportions of islet hormone-positive cells in hPSC-islets detected by flow cytometry (n=6). (e-h) Transplanted hPSC-islets reversed diabetes in STZ-induced diabetic mice. e, Immunofluorescence staining of islet hormones and key markers of β cells in hPSC-islet graft at 16 wpt. Scale bar, 50 μm. f, Fasting blood glucose levels of hPSC-islet transplanted diabetic mice (n=22). g, Human C-peptide secretion in response to glucose challenge in hPSC-islet transplanted mice at 16 wpt (n=17). h, Continuous detection of fasting human C-peptide secretion in hPSC-islet transplanted non-diabetic mice; first detection was conducted at 2 wpt (n=31). All data presented as mean±SEM.

FIG. 2 illustrates intraportal infusion of hPSC-islets led to stabilization of blood glucose levels in immunosuppressed diabetic rhesus macaques. Long-term tracking of glycemic measures in diabetic Monkey-1 #(a, e, i), Monkey-2 #(b, f, j), Monkey-3 #(c, g, k) and Monkey-4 #(d, h, l) pre- and post-infusion of hPSC-islets. (a-d) Daily fasting blood glucose levels of the monkeys pre- and post-infusion of hPSC-islets (infusion procedure conducted at day 0). (e-h) Average pre-meal blood glucose levels of the monkeys pre- and post-infusion of hPSC-islets. P-values reflect statistical significance of change in each group from pre-infusion (−1 month) levels. * P<0.05, ** P<0.005, *** P<0.0005, **** P<0.00005. n.s., not significant. Data presented as mean±SEM. (i-l) Glycated hemoglobin (HbA1c) levels of the monkeys pre-diabetes induction, pre-infusion (0 wpt) and post-infusion of hPSC-islets.

FIG. 3 illustrates hPSC-islet transplanted diabetic macaques showed significant reduction of exogenous insulin requirement and overall increase of body weight. Long-term tracking of exogenous insulin requirements and body weights in diabetic Monkey-1 #(a, e), Monkey-2 #(b, f), Monkey-3 #(c, g) and Monkey-4 #(d, h) pre- and post-infusion of hPSC-islets. (a-d) Weekly average exogenous insulin dose. Exogenous insulin requirement at the last week pre-infusion and at the final week before submission are indicated above bars. Data presented as mean±SEM. (e-h) Tracking of body weights of the monkeys.

FIG. 4 illustrates detection of C-peptide in hPSC-islet transplanted diabetic rhesus macaques. Long-term tracking of C-peptide secretion in diabetic Monkey-1 #(a, e), Monkey-2 #(b, f), Monkey-3 #(c, g) and Monkey-4 #(d, h) pre- and post-infusion of hPSC-islets. (a-d) Random C-peptide levels of the transplanted monkeys pre-diabetes induction, pre-infusion (0 wpt) and post-infusion. (e-h) Fasting and postprandial C-peptide secretion in the transplanted monkeys. All data presented as mean±SEM.

FIG. 5 illustrates establishment of efficient hPSC-islet generation protocol and characterization of hPSC-islets. a, Flow cytometry analysis comparing differentiation efficiencies between planar culture and suspension culture at various stages of the protocol in terms of pancreatic progenitor markers at the end of Stage 4 and β cell markers at the end of Stage 6 (n=5). b, Flow cytometry of β cell marker expression in Stage 6 aggregates without and with addition of small molecules ISX9 and Wnt-C59, individually or in combination at Stage 5, detected at S6D2 (n=4). c, Continuous stage-wise tracking of pancreatic progenitor, endocrine progenitor, and β cell markers by flow cytometry throughout the differentiation protocol (n=3). (d-h) In vitro characterization of stage 6 islet-like aggregates. d, Representative immunostaining of key β cell transcription factors in sectioned Stage 6 aggregates. Scale bar, 50 μm. e, C-peptide secretion of Stage 6 aggregates (n=3) and primary human islets in static glucose stimulation assay (n=2). Glucose stimulation index as indicated above bars. f, Insulin secretion of hPSC-islets in dynamic perifusion assay. g, Electron microscopy depicting ultrastructure of Stage 6 β cell and insulin granules. Scale bar, 2.5 μm. h, Glucagon secretion by hPSC-islets in static glucose stimulation assay. Data obtained from one representative differentiation batch; n=3, technical replicates. (i-l) hPSC-islets ameliorated diabetes and improved overall survival when transplanted into diabetic mice. i, Left: Representative image of nephrectomized kidney showing the hPSC-islet graft beneath the kidney capsule. Scale bar, 0.1 cm. Middle, right: Hematoxylin & eosin (H&E) histology of kidney section, depicting hPSC-islet graft and graft vascularization. Scale bar, 200 μm (middle), 75 μm (right). j, Continuous tracking of body weight of hPSC-islet transplanted diabetic mice (n=22). k, Blood glucose levels in response to IPGTT of healthy (n=8) and STZ-induced diabetic mice groups with (n=17) and without (n=8) hPSC-islet transplantation at 16 wpt. l, Survival rate of STZ-induced diabetic mice groups with and without hPSC-islet transplantation. All data presented as mean±SEM.

FIG. 6 illustrates the established differentiation protocol performed stably across hPSC lines. Similar marker expression pattern and hyperglycemia reversal capacity were observed across three other hPSC lines subject to the established differentiation protocol. a, Representative flow cytometry of pancreatic developmental markers during differentiation showed similar distribution and efficiencies along progressive stages across three other hPSC lines. b, Representative immunofluorescence staining of islet hormones of hPSC-islet sections derived from three other independent hPSC lines. Scale bar, 50 μm. c, Long-term tracking of fasting blood glucose (left) and body weight (right) in diabetic mice transplanted with hPSC-islets derived from three other independent hPSC lines showing consistent reversal of diabetes. d, Long-term tracking of fasting human C-peptide secretion in non-diabetic mice. Data presented as mean±SEM.

### DETAILED DESCRIPTION

Hereinafter, the present disclosure will be described with reference to embodiments shown in the attached drawings. However, it is to be understood that those descriptions are just provided for illustrative purpose, rather than limiting the present disclosure. Further, in the following, descriptions of known structures and techniques are omitted so as not to unnecessarily obscure the concept of the present disclosure.

### Definitions

Otherwise stated, the terms or expressions should have the following definition or meanings.

Pluripotent stem cells (PSCs) are undifferentiated cells defined by their ability, at the single cell level, to both self-renew and differentiate. Stem cells may produce progeny cells, including self-renewing progenitors, non-renewing progenitors, and terminally differentiated cells. Stem cells may be characterized by their ability to differentiate into functional cells of various cell lineages from multiple germ layers (endoderm, mesoderm, and ectoderm).

Differentiation is the process by which an unspecialized (“uncommitted”) or less specialized cell acquires the features of a specialized cell, for example a nerve cell or a muscle cell. A differentiated cell is one that has taken on a more specialized (“committed”) position within the lineage of a cell.

The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

“hPSC-islets” in this context refers to islets including C-peptide positive cells, glucagon positive cells and somatostatin positive cells, which were derived from human pluripotent stem cells, e.g. by the present method.

“Markers”, as used herein, are nucleic acid or polypeptide molecules that are differentially expressed in a cell of interest so that they can be used to indicate a certain state (such as a developmental stage), a characteristic property and/or identity of the cell. In this context, differential expression means an increased level for a positive marker and a decreased level for a negative marker as compared to an undifferentiated cell or a cell at another stage of differentiation. The detectable level of the marker nucleic acid or polypeptide is sufficiently higher or lower in the cells of interest compared to other cells, such that the cell of interest can be identified and distinguished from other cells using any of a variety of methods known in the art.

As used herein, a cell is “positive” for a specific marker, “positive”, or “+” when the specific marker is beyond detection limit and sufficiently significant in the cell. A proper detection limit can be determined by one skilled in the art depending on the testing method. In particular, positive by flow cytometry (“FC”) is usually greater than about 2%. Positive by polymerase chain reaction cytometry (“PCR”) is usually less than or equal to about 35 cycles (Cts).

As used herein, “suspension culture” refers to a culture of cells, single cells or clusters, suspended in medium rather than adhering to a surface in contrast to adherent culture, such as planar culture. One or more culturing stages of the present method can comprise suspension culture or planar culture. For example, a planar culture is conducted at Stage 1, Stage 2 and Stage 3, while a suspension culture is conducted at Stage 4, Stage 5 and Stage 6 of the present method. The culture stages are described in more detail in the following section and examples.

### Culture Method and Culture Medium

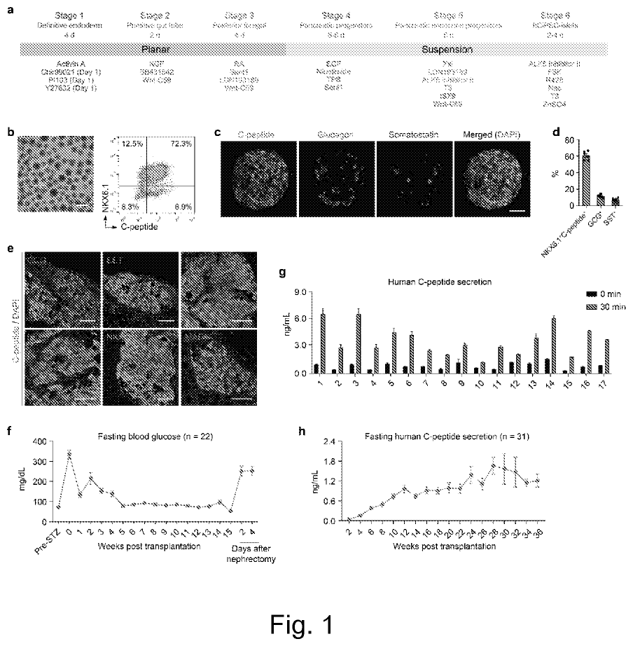

In attempts to replicate the differentiation of pluripotent stem cells into functional pancreatic endocrine cells in vitro cell cultures, the differentiation process is often viewed as progressing through a number of consecutive stages. Referring to FIG. 1(a), in particular, the differentiation process is commonly viewed as progressing through multiple stages, in this step-wise differentiation, “Stage 1” or “Step (1)” refers to the first step in the differentiation process, the differentiation of pluripotent stem cells into cells expressing markers characteristic of the definitive endoderm. “Stage 2” or “Step (2)” refers to the second step, the differentiation of cells expressing markers characteristic of the definitive endoderm cells into cells expressing markers characteristic of primitive gut tube cells. “Stage 3” or “Step (3)” refers to the third step, differentiation of cells expressing markers characteristic of primitive gut tube cells into cells expressing markers characteristic of posterior foregut cells. “Stage 4” or “Step (4)” refers to the fourth step, differentiation of cells expressing markers characteristic of posterior foregut cells into cells expressing markers characteristic of pancreatic progenitors. “Stage 5” or “Step (5)” refers to the fifth step, differentiation of cells expressing markers characteristic of pancreatic progenitors into cells expressing markers characteristic of pancreatic endocrine progenitors. “Stage 6” or “Step (6)” refers to the sixth step, the differentiation of cells expressing markers characteristic of pancreatic endocrine progenitors into cells expressing markers characteristic of functional hPSC-islets.

As used herein “functional hPSC-islets” are islets which are derived from human pluripotent stem cells and possess one or more functional features similar to or same as those of naturally occurring islets in a normal pancreas. One representative function of functional islets is to secrete hormones, such as. The functional hPSC-islets of the present disclosure can be characterized by containing the major pancreatic endocrine cells, including C-peptide⁺ cells, glucagon⁺ cells and somatostatin⁺ cells. Accordingly, the functional hPSC-islets of the present disclosure can be used to treat, alleviate, or reverse a disease or condition resulted from or related to the dysfunction or deficiency of islets of Langerhans, e.g. T1DM. As used herein, the term “treat”, “treating” or “treatment” means the control, reversal or cure of a disease or condition, or one or more symptoms or complications thereof in a subject, including alleviation of a symptom or complication, delay in progression of a disease or condition, or complete cure of a disease or condition. In some embodiments, the term “treat”, “treating” or “treatment” includes those for prophylactic purpose which are administered before the onset or development of a disease or condition, or one or more symptoms or complications thereof. An effective treatment can be determined by measuring physiologic parameters, observing morphology or by any means known in the art or developed in the future for the same purpose. In the context of islet transplantation, an effective treatment can be represented by restoration of endogenous insulin secretion and improvement of glycemic control, which can be characterized by one or more of a decreased fasting blood glucose level, a decreased prepandial blood glucose level, a decreased HbA1c level, a decreased requirement of exogenous insulin, persistent postprandial C-peptide release. For example, according to embodiments of present disclosure, the inventors have demonstrated that transplantation of human pluripotent stem cell-derived islets effectively restored endogenous insulin secretion and improved glycemic control in the recipient. After a one-dose intraportal infusion of stem cell-derived islets, fasting blood glucose and average prepandial blood glucose levels significantly decreased in all recipients. For example, the average HbA1c of the recipient can be reduced by at least 1%, preferably at least 2% after transplantation, e.g. 3 months after transplantation, as compared to the level before transplantation. For example, the average supplement of exogenous insulin is reduced by at least 20%, at least 25%, at least 30%, at least 35%, at least 40%, at least 45%, at least 50% after transplantation, e.g. 3 months after transplantation. In one specific embodiment, three months after transplantation, the average HbA1c dropped by over 2% compared with peak values, while the average exogenous insulin requirement reduced by 46%. Furthermore, persistent C-peptide release in response to meals was observed in all recipients. Notably, these improvements were also observed in a recipient macaque possessing a glycemic status resembling that in patients with labile diabetes, in whom islet replacement therapy confers significant clinical and potentially life-saving benefits.

As used herein, the term diabetes refers to a syndrome that can be characterized by disordered metabolism resulting in abnormally high blood glucose levels (hyperglycemia). The two most common forms of diabetes are due to either a diminished production of insulin (in Type 1), or diminished response by the body to insulin (in Type 2 and gestational). Type 1 diabetes (Type 1 diabetes, Type I diabetes mellitus (T1DM), Insulin dependent diabetes mellitus (IDDM), juvenile diabetes) is a disease that results in the permanent destruction of insulin-producing beta cells of the pancreas. Type 2 diabetes (non-insulin-dependent diabetes mellitus (NIDDM), or adult-onset diabetes) is a metabolic disorder that is primarily characterized by insulin resistance (diminished response by the body to insulin), relative insulin deficiency, and hyperglycemia. Complications associated with diabetes include, but are not limited to hypoglycemia, ketoacidosis, or nonketotic hyperosmolar coma, cardiovascular disease, renal failure, retinal damage, nerve damage, and microvascular damage. In some embodiments, a mammal is pre-diabetic, which can be characterized, for example, as having elevated fasting blood glucose or elevated post-prandial blood glucose.

According to a first aspect of the present disclosure, a method of in vitro generating functional hPSC-islets is provided, and the method comprises:
(1) culturing the hPSCs in a sixth culture medium to obtain cells expressing markers characteristic of the definitive endoderm;
(2) culturing the cells obtained in step (1) in a fifth culture medium to obtain cells expressing markers characteristic of primitive gut tube cells;
(3) culturing the cells obtained in step (2) in a fourth culture medium to obtain cells expressing markers characteristic of posterior foregut cells;
(4) culturing the cells obtained in step (3) in a third culture medium to obtain cells expressing markers characteristic of pancreatic progenitors;
(5) culturing the cells obtained in step (4) in a second culture medium to obtain cells expressing markers characteristic of pancreatic endocrine progenitors;
(6) culturing the cells obtained in step (5) in a first culture medium to obtain cells expressing markers characteristic of functional hPSC-islets;
wherein the second culture medium comprises ISX9, or Wnt-C59.

In a preferred embodiment, the second culture medium comprises ISX9.

In a further embodiment, the second culture medium comprises a small molecule combination of ISX9 and Wnt-C59.

The medium of the present application comprises a basal medium. The term “basal medium” as described herein refers to a composition providing the nutrients such as amino acids, vitamins, carbohydrates and salts which support the survival and growth of cells. In most of the cases, as a basal medium alone is insufficient to support the growth of cells, supplements are usually added to a basal medium to provide cells with compounds which are essential for their growth and/or differentiation. Exemplary basal medium suitable for culturing mammalian stem cells is known in the art, including but not limited to Dulbecco's Modified Eagle Media (DMEM) or DMEM-derived media, e.g. DMEM basic, DMEM/F12, Knockout-DMEM (KO-DMEM), MCBD, RPMI 1640, CMRL 1066 or the like.

Basal medium can be supplemented with nutrients depending on the type of culture cells. The supplemented nutrients can include a commercially available premix, or can be formulated as needed. Exemplary nutritional supplements include but not limited to B27 supplements, FBS (fetal calf serum), BSA, N2 and GlutaMAX.

For example, B27 supplement or BSA is added to a basal medium of any stage in the present application. Preferably, B27 is added in an amount of 0.01-10%, more preferably 0.5-2%, e.g. 0.5%, 0.75%, 1%, 1.5%, 2%, most preferably 1%, or added in an amount as recommended by the manufacturer. In some cases, B27 supplement is interchangeable with BSA or FBS in an equivalent amount.

For example, when MCBD 131 can be added to the medium.

To induce differentiation of cells, one or more differentiating agents can be included in the medium depending on the culture stage. The “differentiating agent” as described herein refers to any agent that facilitates the development from hPSC towards functional hPSC-islets. Differentiating agents in the present application can include growth factors, such as KGF and EGF. A differentiating agent can function by improving or increasing the generation or growth of a desired cell type, and/or inhibiting or decreasing the generation or growth of one or more undesired cell types, by known or unknown mechanisms. The present invention is at least based on an unexpected that the small molecules, specifically ISX9 and/or Wnt-C59, promotes the differentiation from hPSC towards islets, especially during the terminal differentiation of pancreatic endocrine progenitors. FIG. 1a shows a specific embodiment of preferable combination of differentiating agents used in each stage of the culture method of the present invention.

One skilled in the art would also understand that use of a certain type of product is not mandatory in the present application. Any basal medium or nutritional supplements can be substituted with a functional alternative.

### First Culture Medium (Stage 6)

The first culture medium comprises a basal medium supplemented with one or more differentiating agent selected from an ALK inhibitor, an Adenylyl cyclase activator, an Axl inhibitor, an IκB kinase inhibitor, a thyroid hormone and ZnSO₄. (Stage 6) In a preferred embodiment, the first culture medium comprises a basal medium supplemented with a combination of an ALK5 inhibitor, an Adenylyl cyclase activator, an Axl inhibitor, an IκB kinase inhibitor, a thyroid hormone and ZnSO₄.

In some embodiments, the ALK inhibitor can be an ALK5 inhibitor which selectively inhibits ALK5, such as ALK5 inhibitor II, 616452, RepSox (E-616452), SB431542 and A83-01. The ALK inhibitor, e.g. ALK5 inhibitor II, is added in an amount of about 1 to 50 μM, preferably about 5 to 15 μM, or more preferably about 10 μM. In some embodiments, the Adenylyl cyclase activator is Forskolin. The adenylyl cyclase activator, e.g. Forskolin, is added in an amount of about 1 to 100 μM, preferably about 5 to 15 μM, or more preferably about 10 μM.

In some embodiments, the Axl inhibitor is R428 or analog thereof. The Axl inhibitor, e.g. R428, is added in an amount of about 0.1 to 10 μM, preferably about 0.1 to 1 μM, or more preferably about 0.5 M.

In some embodiments, the IκB kinase inhibitor is N-acetyl cysteine or analog thereof. The IκB kinase inhibitor, e.g. N-acetyl cysteine or analog thereof, is added in an amount of about 0.5 to 20 mM, preferably about 1 to 5 mM, or more preferably about 2 mM.

In some embodiments, the thyroid hormone is liothyronine sodium (T3). The thyroid hormone, e.g. T3, is added in an amount of about 0.1 to 20 μM, preferably about 0.5 to 1.5 μM, or more preferably about 1 μM.

In some embodiments, the first culture medium is a basal medium supplemented with ALK5 inhibitor II, Forskolin, R428, N-acetyl cysteine, T3 and ZnSO₄.

In some embodiments, the first culture medium is further supplemented with at least one of B27, heparin, and Vitamin C. B27 in the first culture medium can be replaced by BSA or FBS in a functionally equivalent amount.

In preferred embodiments, the first culture medium comprises, in addition to a basal medium, one or more of about 1 to 50 μM ALK5 inhibitor II, about 0.1 to 10 μM R428, about 0.1 to 20 μM T3, about 1 to 100 M Forskolin, about 1 to 100 μM ZnSO₄, and about 0.5 to 20 mM N-acetyl cysteine.

In preferred embodiments, the first culture medium comprises in addition to a basal medium: about 5 to 15 μM ALK5 inhibitor II, about 0.1 to 1 μM R428, about 0.5 to 1.5 μM T3, about 5 to 15 μM Forskolin, about 5 to 15 μM ZnSO₄, and/or about 1 to 5 mM N-acetyl cysteine.

In some embodiments, the first culture medium comprises, in addition to a basal medium: about 0.5% to 2% B27, about 5 to 15 μM ALK5 inhibitor II, about 0.1 to 1 μM R428, about 0.5 to 1.5 μM T3, about 5 to 15 μM Forskolin, about 5 to 15 μg/mL heparin, about 5 to 15 μM ZnSO₄, about 1 to 5 mM N-acetyl cysteine, and/or about 0.1 to 0.5 mM Vitamin C.

In some embodiments, the first culture medium comprises: about 1% B27, about 10 μM ALK5 inhibitor II, about 0.5 μM R428, about 1 μM T3, about 10 μM Forskolin, about 10 μg/mL heparin, about 10 μM ZnSO₄, about 2 mM N-acetyl cysteine, and/or about 0.25 mM Vitamin C.

In a preferred embodiment, the culture at Stage 6 of the present method is conducted in suspension for about 2 to 6 days by using the first culture medium to generate islets from pancreatic endocrine progenitors.

### Second Culture Medium (Stage 5)

The pancreatic endocrine progenitor cells are obtained by culturing pancreatic progenitor cells in a second culture medium comprising Isoxazole 9 (ISX9) or preferably a combination of ISX9 and an inhibitor of Wnt signaling as differentiating agent (Stage 5).

ISX9 is added in an amount of about 0.5 to 200 μM, preferably about 0.5 to 100 μM, more preferably about 10 μM.

The inhibitor of Wnt signaling, e.g. Wnt-C59, is added in an amount of about 5 to 2000 nM, more preferably about 10 to 500 nM, more preferably about 50 to 250 nM, or even more preferably about 100 nM.

In some embodiments, the second culture medium is further supplemented with one or more differentiating agent selected from a group consisting of an ALK inhibitor, a BMP signaling inhibitor, a thyroid hormone and an inhibitor of NOTCH signaling. (Stage 5)

For example, the inhibitor of ALK inhibitor is an ALK5 inhibitor can be an ALK5 inhibitor which selectively inhibits ALK5, such as ALK5 inhibitor II, 616452, RepSox (E-616452), SB431542 and A83-01. The ALK inhibitor, e.g. ALK5 inhibitor II, is added in an amount of about 1 to 50 μM, preferably about 5 to 15 μM, or more preferably about 10 μM.

For example, the BMP (bone morphogenetic proteins) signaling inhibitor is LDN193189. The BMP signaling inhibitor, e.g. LDN193189, is added in an amount of about 0.015 to 10 M, preferably about 0.05 to 5 μM, more preferably about 0.1 to 1 μM, or even more preferably about 0.3 μM.

For example, the thyroid hormone is T3. The thyroid hormone, e.g. T3, is added in an amount of about 0.05 to 50 μM, preferably about 0.1 to 10 μM, more preferably 0.5 to 5 μM, even more preferably about 1 μM.

For example, the inhibitor of NOTCH signaling is Xxi. The inhibitor of NOTCH signaling, e.g. Xxi, is added in an amount of about 0.005 to 2 μM, preferably about 0.05 to 1 μM, or more preferably about 0.1 μM.

In some embodiments, the second culture medium is further supplemented with a ROCK inhibitor, such as Y27632. The ROCK inhibitor, such as Y27632, is added in an amount of about 0.5 to 200 μM, preferably about 0.4 to 100 μM, or more preferably about 10 μM.

In some embodiments, the second culture medium is further supplemented with one or more of L-glutamine such as Glutamax, B27, heparin, and Vitamin C.

In some embodiments, the basal medium of the second culture medium is DMEM basic medium or MCBD medium.

In some embodiments, the second culture medium comprises in addition to basal medium: about 0.01% to 10% B27, about 0.5 to 200 μM ALK5 inhibitor II, about 0.015 to 6 μM LDN193189, about 0.05 to 20 μM T3, about 0.5 to 200 μM ISX9, about 0.5 to 200 g/mL heparin, about 0.005 to 2 μM notch inhibitor Xxi, about 5 to 2000 nM Wnt-C59, about 0.5 to 200 μM Y27632, and about 0.0125 to 5 mM Vitamin C; and optionally about 0.05% to 20% Glutamax.

In some embodiments, the second culture medium comprises in addition to basal medium: preferably 1% Glutamax, about 0.025x to 5x B27, about 0.5 to 100 μM ALK5 inhibitor II, about 0.015 to 10 μM LDN193189, about 0.05 to 50 μM T3, about 0.5 to 100 μM ISX9, about 0.5 to 100 g/mL heparin, about 0.005 to 2 μM y-secretase inhibitor Xxi, about 5 to 2000 nM Wnt-C59, about 0.5 to 200 μM Y27632, and/or about 0.0125 to 5 mM Vitamin C; and optionally about 0.05% to 5% Glutamax.

In some embodiments, the second culture medium comprises in addition to basal medium: about 1% B27, about 10 μM ALK5 inhibitor II, about 0.3 μM LDN193189, about 1 μM T3, about 10 μM ISX9, about 10 μg/mL heparin, about 0.1 μM γ-secretase inhibitor Xxi, about 100 nM Wnt-C59, about 10 μM Y27632, and/or about 0.25 mM Vitamin C; and optionally about 1% Glutamax.

In a preferred embodiment, the culture at Stage 5 of the present method is conducted in suspension for about 3 to 10 days, particularly about 4 to 6 days by using the second culture medium to generate pancreatic endocrine progenitors from pancreatic progenitors.

### Third Culture Medium (Stage 4)

In some embodiments, the pancreatic progenitor cells are obtained by culturing posterior foregut in a third culture medium comprising a basal medium supplemented with one or more differentiating agents selected from a group consisting of a growth factor such as epidermal growth factor (EGF), a B-Complex Vitamin such as Nicotinamide, an activator of protein kinase C such as TPB, and an inhibitor of Sonic hedgehog signaling such as Sant1. (Stage 4)

In some embodiments, the pancreatic progenitor cells are obtained by culturing posterior foregut in a third culture medium supplemented with one or more of EGF, Nicotinamide, TPB, and Sant1, preferably a combination of EGF, Nicotinamide, TPB, and Sant1.

For example, the growth factor, e.g. EGF, is added in an amount of about 1 to 2000 ng/ml, preferably about 5 to 500 ng/ml, more preferably about 10 to 200 ng/ml, or even more preferably 100 ng/ml.

For example, the B-Complex Vitamin, e.g. nicotinamide, is added in an amount of about 0.5 to 200 mM, preferably about 1 to 100 mM, more preferably about 5 to 50 mM, or even more preferably about 10 mM.

For example, the activator of protein kinase C, e.g. TPB, is added in an amount of about 0.01 to 10 μM, preferably about 0.02 to 5 μM, more preferably about 0.1 to 1 μM, or even more preferably about 0.2 μM.

For example, the inhibitor of Sonic hedgehog signaling, e.g. Sant1, is added in an amount of about 0.0125 to 5 μM, preferably about 0.025 to 1 μM, more preferably about 0.05 to 0.5 μM, or even more preferably about 0.25 μM.

In some embodiments, the third culture medium is further supplemented with one or more of L-glutamine such as Glutamax, B27, and Vitamin C.

In some embodiments, the basal medium of the third culture medium is DMEM basic medium.

In some embodiments, the third culture medium comprises in addition to a basal medium: about 0.01% to 10% B27, about 5 to 2000 ng/ml EGF, about 0.01 to 4 μM TPB, about 0.5 to 200 mM Nicotinamide, about 0.0125 to 5 μM Sant1 and about 0.0125 to 5 mM Vitamin C; and optionally about 0.05% to 20% Glutamax. In some embodiments, the third culture medium comprises in addition to a basal medium: about 0.01% to 10% B27, about 1 to 500 ng/ml EGF, about 0.01 to 10 μM TPB, about 0.5 to 200 mM Nicotinamide, about 0.0125 to 5 μM Sant1 and/or about 0.0125 to 5 mM Vitamin C; and optionally about 0.05% to 20% Glutamax. In some embodiments, the third culture medium comprises in addition to a basal medium: about 1% B27, about 100 ng/ml EGF, about 0.2 μM TPB, about 10 mM Nicotinamide, about 0.25 μM Sant1 and/or about 0.25 mM Vitamin C; and optionally about 1% Glutamax.

In a preferred embodiment, the culture at Stage 4 of the present method is conducted in suspension for about 4 to 7 days, preferably 5 to 6 days by using the third culture medium to generate pancreatic progenitors from posterior foregut.

### Fourth Culture Medium (Stage 3)

In some embodiments, the posterior foregut is obtained by culturing primitive gut tube in a fourth culture medium comprising a basal medium supplemented with and an inhibitor of Wnt signaling such as Wnt-C59 as differentiating agent. (Stage 3) The inhibitor of Wnt signaling, e.g. Wnt-C59, is added in an amount of about 5 to 2000 nM, more preferably about 10 to 500 nM, more preferably about 50 to 250 nM, or even more preferably about 100 nM.

In further embodiments, the fourth medium is further supplemented with one or more differentiating agents selected from a group consisting of Retinoic acid (RA), an inhibitor of Sonic hedgehog signaling such as Sant1, and an inhibitor of BMP signaling such as LDN193189.

In some embodiments, the posterior foregut is obtained by culturing primitive gut tube in a fourth culture medium supplemented with Wnt-C59 and one or more of Retinoic acid (RA), Sant1, and LDN193189, preferably a combination of Retinoic acid (RA), Sant1, LDN193189 and Wnt-C59.

For example, retinoic acid (RA) is added in an amount of about 0.1 to 40 μM, preferably about 0.5 to 10 μM, more preferably about 1 to 5 μM, or even more preferably about 2 μM.

For example, the inhibitor of Sonic hedgehog signaling, e.g. Sant1, is added in an amount of about 0.0125 to 5 μM, preferably about 0.025 to 1 μM, more preferably about 0.05 to 0.5 μM, or even more preferably about 0.25 μM.

For example, the BMP signaling inhibitor, e.g. LDN193189, is added in an amount of about 0.01 to 2 μM, preferably about 0.05 to 1 μM, or more preferably about 0.1 μM.

In some embodiments, the basal medium of the fourth culture medium is DMEM basic medium.

In some embodiments, the fourth culture medium comprises in addition to a basal medium: about 0.01% to 10% B27, about 0.1 to 40 μM Retinoic acid, about 0.05 to 2 μM LDN193189, about 0.0125 to 5 μM Sant1 and/or about 5 to 2000 nM Wnt-C59.

In some embodiments, the fourth culture medium comprises in addition to a basal medium: about 1% B27, about 2 μM Retinoic acid, about 0.1 μM LDN193189, about 0.25 μM Sant1 and/or about 100 nM Wnt-C59.

In a preferred embodiment, the culture at Stage 3 of the present method is conducted for about 2 to 7 days, e.g. 2, 3, 4, 5, 6 or 7 days, by using the fourth culture medium to generate posterior foregut from primitive gut tube.

### Fifth Culture Medium (Stage 2)

In some embodiments, the primitive gut tube is obtained by culturing a definitive endoderm in a fifth culture medium comprising a basal medium supplemented with one or more differentiating agents selected from a group consisting of a fibroblast growth factor such as KGF, FGF2 and/or FGF10, a TGF-beta/Smad inhibitor such as SB431542, and/or a Wnt inhibitor such as Wnt-C59. (Stage 2) In some embodiments, the primitive gut tube is obtained by culturing a definitive endoderm in a fifth culture medium supplemented with KGF.

In some embodiments, the primitive gut tube is obtained by culturing a definitive endoderm in a fifth culture medium supplemented with KGF, SB431542, and Wnt-C59.

For example, the growth factor, e.g. KGF, is added in an amount of about 2.5 to 1000 ng/ml, preferably about 5 to 500 ng/ml, more preferably about 10 to 100 ng/ml, or even more preferably about 50 ng/ml.

For example, the TGF-beta/Smad inhibitor, e.g. SB431542, is added in an amount about 0.25 to 100 μM, preferably about 0.5 to 50 μM, more preferably about 1 to 10 μM, or even more preferably about 5 μM.

The inhibitor of Wnt signaling, e.g. Wnt-C59, is added in an amount of about 5 to 2000 nM, more preferably about 10 to 500 nM, more preferably about 50 to 250 nM, or even more preferably about 100 nM.

In some embodiments, the basal medium of the fifth culture medium is MCBD 131 medium.

In some embodiments, the fifth culture medium comprises in addition to a basal medium: about 0.225 to 90 mM Glucose, about 0.025% to 10% BSA or 0.01% to 10% B27, about 2.5 to 1000 ng/ml KGF, about 0.0125 to 5 mM Vitamin C, about 0.25 to 100 μM SB431542 and/or about 5 to 2000 nM Wnt-C59; and optionally about 0.05% to 20% Glutamax.

In some embodiments, the fifth culture medium comprises in addition to a basal medium: about 4.5 mM Glucose, about 0.5% BSA or 1% B27, about 50 ng/mL KGF, about 0.25 mM Vitamin C, about 5 μM SB431542 and/or about 100 nM Wnt-C59; and optionally about 1% Glutamax.

In a preferred embodiment, the culture at Stage 2 of the present method is conducted for about 1 to 4 days, e.g. 1, 2, 3, or 4 day(s), by using the fifth culture medium to generate primitive gut tube from definitive endoderm.

### Sixth Culture Medium (Stage 1)

In some embodiments, the method comprises culturing the pluripotent stem cell in the sixth culture medium comprising a basal medium supplemented with one or more differentiating agent selected from a group consisting of Activin A, a Wnt activator such as Chir99021, a PI3K inhibitor such as PI103, and an ROCK inhibitor such as Y27632, and optionally in a seventh culture medium comprising a basal medium supplemented with Activin A.

For example, Activin A in the sixth or seventh medium is added in an amount of about 20-1000 ng/ml, preferably about 50-500 ng/ml, or more preferably about 100 ng/ml.

For example, the Wnt activator, e.g. Chir99021, is added in an amount of about 0.3 to 120 M, preferably 1 to 60 μM, more preferably 3 to 20 μM or even more preferably about 6 μM.

For example, the PI3K inhibitor, e.g. PI103, is added in an amount of about 2.5 to 1000 nM, preferably 5 to 500 nM, more preferably 10 to 100 nM or even more preferably about 50 nM.

For example, the ROCK Inhibitor, e.g. Y27632, is added in an amount of about 0.5 to 200 μM, preferably about 0.4 to 100 μM, or more preferably about 10 μM.

In a further embodiment, the sixth culture medium further comprises one or more of Glucose, Glutamax, B27, and Vitamin C.

In some embodiments, the basal medium of the sixth culture medium is MCBD 131 medium.

In some embodiments, the definitive endoderm is obtained by culturing pluripotent stem cell in a sixth culture medium supplemented with about 0.225 to 90 mM Glucose, about 0.01% to 10% B27, about 20 to 1000 ng/ml Activin A, about 0.0125 to 5 mM Vitamin C, about 0.3 to 120 μM Wnt activator, about 2.5 to 1000 nM PI3K inhibitor and/or about 0.5 to 200 μM ROCK Inhibitor; and optionally about 0.05% to 20% Glutamax.

In another further embodiment, the seventh culture medium further comprises Glucose, Glutamax, B27, and Vitamin C.

In some embodiments, the basal medium of the seventh culture medium is MCBD 131 medium.

In some embodiments, the method comprises culturing the pluripotent stem cell in the sixth culture medium supplemented with about 4.5 mM Glucose, about 1% Glutamax, about 1% B27, about 100 ng/ml Activin A, about 0.25 mM Vitamin C, about 6 μM Chir99021, about 50 nM PI103 and/or about 10 μM Y27632 for about 1 day, and then in the seventh culture medium supplemented with about 0.225 to 90 mM Glucose, about 0.05% to 20% Glutamax, about 0.01% to 10% B27, about 5 to 2000 ng/ml Activin A, and/or about 0.0125 to 5 mM Vitamin C for about 2-4 days.

In some embodiments, the method comprises culturing the pluripotent stem cell in the sixth culture medium supplemented with about 4.5 mM Glucose, about 1% Glutamax, about 1% B27, about 100 ng/ml Activin A, about 0.25 mM Vitamin C, about 6 μM Chir99021, about 50 nM PI103 and/or about 10 μM Y27632 for about 1 day, and then in the seventh culture medium supplemented with about 4.5 mM Glucose, about 1% Glutamax, about 1% B27, about 100 ng/ml Activin A, and/or about 0.25 mM Vitamin C for about 3 days.

In some embodiments, the pluripotent stem cell can be embryonic stem cell, or induced pluripotent stem cell, such as chemically induced pluripotent stem cell. The embryonic stem cells can be commercially available embryonic stem cells. The embryonic stem cells can be derived from in vitro-fertilized embryos. The embryonic stem cells can be obtained from embryos that have not been developed in vivo and are within 14 days after fertilization.

In a specific embodiment, the method comprises: (1) culturing the pluripotent stem cell in the sixth culture medium supplemented with about 4.5 mM Glucose, about 1% Glutamax, about 1% B27, about 100 ng/ml Activin A, about 0.25 mM Vitamin C, about 6 μM Chir99021, about 50 nM PI103 and about 10 μM Y27632 for about 1 day, and then in the seventh culture medium supplemented with about 4.5 mM Glucose, about 1% Glutamax, about 1% B27, about 100 ng/ml Activin A, about 0.25 mM Vitamin C for about 3 days to obtain the definitive endoderm; (2) culturing the definitive endoderm in the fifth culture medium comprising about 4.5 mM Glucose, about 1% Glutamax, about 1% B27, about 50 ng/ml KGF, about 0.25 mM Vitamin C, about 5 μM SB431542 and about 100 nM Wnt-C59 for about 2 days to obtain the primitive gut tube; (3) culturing the primitive gut tube in the fourth culture medium comprising about 1% B27, about 2 μM Retinoic acid, about 0.1 μM LDN193189, about 0.25 μM Sant1 and about 100 nM Wnt-C59 for about 4 days to obtain the posterior foregut; (4) suspension culturing posterior foregut in the third culture medium comprising about 1% Glutamax, about 1% B27, about 100 ng/ml EGF, about 0.2 μM TPB, about 10 mM Nicotinamide, about 0.25 μM Sant1 and about 0.25 mM Vitamin C for about 5 to 6 days to obtain the pancreatic progenitor cells; (5) suspension culturing the pancreatic progenitor cells in the second culture medium comprising about 1% Glutamax, about 1% B27, about 10 μM ALK5 inhibitor II, about 0.3 μM LDN193189, about 1 μM T3, about 10 μM ISX9, about 10 μg/mL heparin, about 0.1 μM γ-secretase inhibitor Xxi, about 100 nM Wnt-C59, about 10 μM Y27632, and about 0.25 mM Vitamin C for about 6 days to obtain the pancreatic endocrine progenitor cells; and (6) suspension culturing the pancreatic endocrine progenitor cells in the first culture comprising about 1% B27, about 10 μM ALK5 inhibitor II, about 0.5 μM R428, about 1 μM T3, about 10 μM Forskolin, about 10 μg/mL heparin, about 10 μM ZnSO₄, about 2 mM N-acetyl cysteine, and about 0.25 mM Vitamin C medium for about 2 to 4 days to obtain the functional hPSC-islets that contain C-peptide⁺ cells, glucagon⁺ cells and somatostatin⁺ cells. In a preferred embodiment of the first aspect, the method generates functional hPSC-islets that contain C-peptide⁺ cells, glucagon⁺ cells and somatostatin⁺ cells. According to embodiments, the method produces relatively uniform, islet-sized aggregates containing NKX6.1+C-peptide⁺ cells at an efficiency of up to approximately 70% from human pluripotent stem cells (hPSCs). Dynamic analysis of the differentiation process showed that approximately 90% PDX1⁺ pancreatic progenitors were generated by early Stage 4, which finally gave rise to 90% CHGA⁺NGN3⁻ endocrine cells in Stage 6. Notably, the protocol showed stable performance, consistently reproducing similar results across differentiation batches. Collectively, these data indicated the establishment of a protocol that robustly promoted pancreatic endocrine differentiation from hPSCs.

hPSC-Islets

According to a second aspect of the present disclosure, a population of functional hPSC-islets obtainable by the method described above are provided. These population of functional hPSC-islets may be used to treat a mammal having, or at risk of having, type I diabetes, type II diabetes, pre-diabetes or any combination thereof, for example by transplanting these islets in a subject in need of such treatment.

According to a third aspect of the present disclosure, a pharmaceutical composition comprising the population of functional hPSC-islets described above. The pharmaceutical composition may be used to treat a mammal having, or at risk of having, type I diabetes, type II diabetes, pre-diabetes or any combination thereof, for example by transplanting these islets in a subject in need of such treatment. According to a fourth aspect of the present disclosure, there is provided with a method for treating a mammal having, or at risk of having, type I diabetes, type II diabetes, pre-diabetes or any combination thereof, the method comprising administering to the mammal the population of functional hPSC-islets described above or the pharmaceutical composition described above.

According to a fifth aspect of the present disclosure, there is provided with a kit for generating functional hPSC-islets, comprising: at least one of a first to a sixth culture medium described above.

According to a sixth aspect of the present disclosure, there is provided with a combination of small molecules ISX9 and Wnt-C59 in inducing differentiation of pancreatic progenitors into pancreatic endocrine progenitors.

### Embodiments of the Present Invention

1. A method of in vitro generating functional hPSC-islets that contain C-peptide⁺ cells, glucagon⁺ cells and somatostatin⁺ cells, comprising:
(1) culturing the hPSCs in a sixth culture medium to obtain cells expressing markers characteristic of the definitive endoderm;
(2) culturing the cells obtained in step (1) in a fifth culture medium to obtain cells expressing markers characteristic of primitive gut tube;
(3) culturing the cells obtained in step (2) in a fourth culture medium to obtain cells expressing markers characteristic of posterior foregut;
(4) culturing the cells obtained in step (3) in a third culture medium to obtain cells expressing markers characteristic of pancreatic progenitors;
(5) culturing the cells obtained in step (4) in a second culture medium to obtain cells expressing markers characteristic of pancreatic endocrine progenitors;
(6) culturing the cells obtained in step (5) in a first culture medium to obtain cells expressing markers characteristic of functional hPSC-islets;
wherein the second culture medium is supplemented with ISX9 or Wnt-C59, preferably ISX9.

2. The method of claim 1, wherein the second culture medium is supplemented with ISX9 and Wnt-C59.

3. The method of embodiment 1 or embodiment 2, wherein the first culture medium comprises a basal medium supplemented with one or more of an ALK5 inhibitor, an Adenylyl cyclase activator, an Axl inhibitor, an IκB kinase inhibitor, T3 and ZnSO₄.

4. The method of embodiment 3, wherein the ALK5 inhibitor is ALK5 inhibitor II or analog thereof.

5. The method of embodiment 3 or embodiment 4, wherein the Adenylyl cyclase activator is Forskolin or analog thereof.

6. The method of any one of embodiments 3 to 5, wherein the Axl inhibitor is R428 or analog thereof.

7. The method of any one of embodiments 3 to 6, wherein the IκB kinase inhibitor is N-acetyl cysteine or analog thereof.

8. The method of any one of embodiments 1 to 7, wherein the basal medium of the first culture medium is a DMEM basic or MCBD131.

9. The method of any one of embodiments 1 to 8 wherein the first culture medium is further supplemented with one or more of B27, heparin, and Vitamin C.

10. The method of any one of embodiments 1 to 9, wherein the second culture medium comprises a basal medium further supplemented with one or more of an inhibitor of TGF-βRI, a BMP signaling inhibitor, a thyroid hormone and an inhibitor of NOTCH signaling.

11. The method of embodiment 10, wherein the inhibitor of TGF-βRI is ALK5 inhibitor II.

12. The method of embodiment 10 or embodiment 11, wherein the BMP signaling inhibitor is LDN193189.

13. The method of any one of embodiments 10 to 12, wherein the thyroid hormone is T3.

14. The method of any one of embodiments 10 to 13, wherein the inhibitor of NOTCH signaling is γ-secretase inhibitor, such as Xxi.

15. The method of any one of embodiments 10 to 14, wherein the basal medium of the second culture medium is a DMEM basic or MCBD131.

16. The method of any one of embodiments 10 to 15, wherein the second culture medium is further supplemented with one or more of L-glutamine, B27, heparin, Y27632, and Vitamin C.

17. The method of any one of embodiments 1 to 16, wherein the third culture medium comprises a basal medium supplemented with one or more of an epithelial growth factor, an activator of protein kinase C, an inhibitor of Sonic hedgehog signaling and a component of the vitamin B complex.

18. The method of embodiment 17, wherein the epithelial growth facture is EGF.

19. The method of embodiment 17 or embodiment 18, wherein the activator of protein kinase C is TPB.

20. The method of any one of embodiments 17 to 19, wherein the inhibitor of Sonic hedgehog signaling Sant1.

21. The method of any one of embodiments 17 to 20, wherein the component of the vitamin B complex is Nicotinamide.

22. The method of any one of embodiments 17 to 21, wherein the basal medium of the third culture medium is a DMEM basic or MCBD131.

23. The method of any one of embodiments 17 to 22, wherein the third culture medium is further supplemented with one or more of L-glutamine, B27, and Vitamin C.

24. The method of any one of embodiments 1 to 23, wherein the fourth culture medium is supplemented with one or more of Retinoic acid (RA), an inhibitor of Sonic hedgehog signaling, and an inhibitor of BMP signaling.

25. The method of embodiment 24, wherein the inhibitor of Sonic hedgehog signaling is Sant1.

26. The method of embodiment 24 or embodiment 25, wherein the inhibitor of BMP signaling is LDN193189.

27. The method of any one of embodiments 24 to 26, wherein the fourth culture medium is further supplemented with an inhibitor of Wnt signaling.

28. The method of embodiment 27, wherein the inhibitor of Wnt signaling is Wnt-C59.

29. The method of any one of embodiments 24 to 28, wherein the basal medium of the fourth culture medium is a DMEM basic or MCBD131.

30. The method of any one of embodiments 24 to 29, wherein the fourth medium is further supplemented with B27.

31. The method of any one of embodiments 1 to 30, wherein the fifth culture medium comprises a basal medium supplemented with an activator of FGF signaling.

32. The method of embodiment 31, wherein the activator of FGF signaling is FGF2, FGF10 or KGF.

33. The method of embodiment 31 or embodiment 32, wherein the fifth culture medium is further supplemented with a TGF-beta/Smad inhibitor, and/or a Wnt inhibitor.

34. The method of any one of embodiment 33, wherein the TGF-beta/Smad inhibitor is SB431542.

35. The method of embodiment 33 or 34, wherein the Wnt inhibitor is Wnt-C59.

36. The method of any one of embodiments 31 to 35, wherein the basal medium of the fifth culture medium is DMEM basic or MCBD 131.

37. The method of any one of embodiments 31 to 36, wherein the fifth culture medium is further supplemented with one or more of Glucose, L-glutamine, B27 or BSA, KGF, and Vitamin C.

38. The method of any one of embodiments 1 to 36, wherein the sixth culture medium comprises a basal medium supplemented with one or more of an activator of Activin receptor, a Wnt activator, a ROCK inhibitor and an PI3K inhibitor.

39. The method of embodiment 38, wherein the activator of Activin receptor is Activin A.

40. The method of embodiments 38 to 39, wherein the Wnt activator is Chir99021.

41. The method of any one of embodiments 38 to 40, wherein the ROCK inhibitor is Y27632.

42. The method of any one of embodiments 38 to 41, wherein the PI3K inhibitor is PI103.

43. The method of any one of embodiments 38 to 42, wherein the basal medium of the sixth culture medium is DMEM basic or MCBD 131 medium.

44. The method of any one of embodiments 38 to 43, wherein the sixth culture medium is further supplemented with one or more of Glucose, L-glutamine, B27, Vitamin C.

45. The method of any one of embodiments 1 to 44, wherein step (1) further comprises culturing in the seventh culture medium after culturing in the sixth culture medium and before step (2), wherein the seventh culture medium comprises a basal medium supplemented with Glucose, L-glutamine, B27, Activin A, and Vitamin C.

46. The method of embodiment 45, comprising culturing the pluripotent stem cells in the sixth culture medium supplemented with about 4.5 mM Glucose, about 1% Glutamax, about 1% B27, about 100 ng/ml Activin A, about 0.25 mM Vitamin C, about 6 μM Chir99021, about 50 nM PI103 and about 10 μM Y27632 for about 1 day, followed by culturing the cells in the seventh culture medium supplemented with about 4.5 mM Glucose, about 1% Glutamax, about 1% B27, about 100 ng/ml Activin A, about 0.25 mM Vitamin C for about 3 days.

47. The method of any one of embodiments 1 to 46, wherein the human pluripotent stem cells are embryonic stem cells or induced pluripotent stem cells.

48. The method of any one of embodiments 1 to 47, the culture of one or more of steps (1) to (6) is suspension culture.

49. The method of embodiment 48, the culture of step (1) to step (3) is suspension culture.

50. A population of cells comprising functional hPSC-islets obtainable by the method of any one of embodiments 1 to 49.

51. A pharmaceutical composition comprising the population of cells of embodiment 50.

52. A method for treating a mammal having, or at risk of having, type I diabetes, type II diabetes, pre-diabetes or any combination thereof, the method comprising administering to the mammal the population of cells of embodiment 50 or the pharmaceutical composition of embodiment 51.

53. A kit for generating functional hPSC-islets that contain C-peptide⁺ cells, glucagon⁺ cells and somatostatin⁺ cells, comprising:
at least one of a first to a seventh culture medium defined in any one of embodiments 1 to 49.

54. A method of in vitro generating functional hPSC-islets that contain C-peptide⁺ cells, glucagon⁺ cells and somatostatin⁺ cells, comprising:
culturing the pluripotent stem cell in the sixth culture medium supplemented with about 4.5 mM Glucose, about 1% Glutamax, about 1% B27, about 100 ng/ml Activin A, about 0.25 mM Vitamin C, about 6 μM Chir99021, about 50 nM PI103 and about 10 μM Y27632 for about 1 day, and then in the seventh culture medium supplemented with about 4.5 mM Glucose, about 1% Glutamax, about 1% B27, about 100 ng/mL Activin A, about 0.25 mM Vitamin C for about 3 days to obtain the definitive endoderm;
culturing the definitive endoderm in the fifth culture medium comprising about 4.5 mM Glucose, about 1% Glutamax, about 0.5% BSA or 1% B27, about 50 ng/ml KGF, about 0.25 mM Vitamin C, about 5 μM SB431542 and about 100 nM Wnt-C59 for about 2 days to obtain the primitive gut tube;
comprising culturing the primitive gut tube in the fourth culture medium comprising about 1% B27, about 2 μM Retinoic acid, about 0.1 μM LDN193189, about 0.25 μM Sant1 and about 100 nM Wnt-C59 for about 4 days to obtain the posterior foregut;
culturing posterior foregut in the third culture medium comprising about 1% Glutamax, about 1% B27, about 100 ng/ml EGF, about 0.2 μM TPB, about 10 mM Nicotinamide, about 0.25 μM Sant1 and about 0.25 mM Vitamin C for about 5 to 6 days to obtain the pancreatic progenitor cells;
culturing the pancreatic progenitor cells in the second culture medium comprising about 1% Glutamax, about 1% B27, about 10 μM ALK5 inhibitor II, about 0.3 μM LDN193189, about 1 μM T3, about 10 μM ISX9, about 10 μg/mL heparin, about 0.1 μM γ-secretase inhibitor Xxi, about 100 nM Wnt-C59, about 10 μM Y27632, and about 0.25 mM Vitamin C for about 6 days to obtain the pancreatic endocrine progenitor cells;
culturing the pancreatic endocrine progenitor cells in the first culture comprising about 1% B27, about 10 M ALK5 inhibitor II, about 0.5 μM R428, about 1 μM T3, about 10 μM Forskolin, about 10 μg/mL heparin, about 10 μM ZnSO₄, about 2 mM N-acetyl cysteine, and about 0.25 mM Vitamin C medium for about 2 to 6 days to obtain the functional hPSC-islets that contain C-peptide⁺ cells, glucagon⁺ cells and somatostatin⁺ cells.

55. Use of ISX9 and Wnt-C59 in inducing differentiation of pancreatic progenitors into pancreatic endocrine progenitors.

### Examples

The following examples illustrate the present invention, and are set forth to aid in the understanding of the invention, and should not be construed to limit in any way the scope of the invention as defined in the claims which follow thereafter.

### Methods

### Cell Sources and Culture

Home-made human pluripotent stem cells (hPSCs) via chemical reprogramming of fibroblasts were cultured in mTeSR1 (Stem Cell, Cat #85850) on 1:40 diluted Matrigel-coated (BD BioSciences, Cat #356231) plate or dish. Medium was changed daily. Cultures were passaged by ReleSR (Stem Cell, Cat #05872) at a 1:10-1:15 split ratio every 5-6 days.

In Vitro Differentiation to Generate hPSC-Islets

Before differentiation, adherent hPSCs were dispersed into single cells using Accutase (EMD Millipore, Cat #SCR005), rinsed with DMEM/F12 (Gibco, Cat #11330-032) and seeded on Matrigel-coated plate or dich in mTESR1 supplemented with 10 mM Y27632. Differentiation was initiated 24 h following seeding. The detailed information of small molecules and cytokines used in the differentiation process is listed in Table 1.

TABLE 1
Small molecules and cytokines used in differentiation protocol.

| Name | Source | Cat# |
|---|---|---|
| Activin A | Stemimmune LLC | HST-A-1000 |
| KGF | Stemimmune LLC | HST-F7-1000 |
| EGF | Peprotech | AF-100-15 |
| Y27632 | Selleck | S1049 |
| Chir99021 | Selleck | S1263 |
| PI103 | Selleck | S1038 |
| Vitamin C | Sigma | 49752 |
| SB431542 | Selleck | S1067 |
| Wnt-C59 | Selleck | S7037 |
| LDN193189 | Selleck | S7507 |
| Retinoic acid (RA) | Sigma | R2625 |
| Sant1 | Selleck | S7092 |
| Nicotinamide | Sigma | N0636 |
| TPB | Santacruz | SC-204424 |
| ALK5 inhibitor II | Selleck | S7223 |
| Liothyronine Sodium (T3) | Selleck | S4217 |
| Isoxazole 9 (ISX9) | Selleck | S7914 |
| γ-Secretase Inhibitor XX | Calbiochem | 565789 |
| (Xxi) | | |
| R428 | Selleck | S2841 |
| N-Acetyl-L-cysteine (Nac) | Sigma | A9165 |
| Forskolin (FSK) | Selleck | S2449 |
| ZnSO4 | Sigma | Z0251 |
| Heparin sodium | Selleck | S1346 |

Medium formulation at each stage as follows (percentage is calculated by volume unless being indicated otherwise):

Stage 1 (4 days). MCDB131 (Gibco, Cat #10372-019) supplied with 4.5 mM Glucose (Sigma, Cat #G7021), 1% Glutamax (Gibco, Cat #35050-061), 1% Pen/Strep, 1% B27 (Gibco, Cat #12587-010), 100 ng/mL Activin A, 0.25 mM Vitamin C, 6 μM Chir99021, 50 nM PI103 and 10 μM Y27632 for day 1 only. For days 2-4, culture medium was refreshed every day in MCDB131 with 4.5 mM Glucose, 1% Glutamax, 1% Pen/Strep, 1% B27, 50 ng/ml Activin A and 0.25 mM Vitamin C.

Stage 2 (2 days). MCDB131 supplied with 4.5 mM Glucose, 1% Glutamax, 1% Pen/Strep, 0.5% BSA (Sigma, Cat #A4612) or 1% B27, 50 ng/ml KGF, 0.25 mM Vitamin C, 5 M SB431542 and 100 nM Wnt-C59.

Stage 3 (4 days). DMEM-basic (Gibco, Cat #C11965500BT) supplied with 1% Pen/Strep, 1% B27, 2 μM Retinoic acid, 0.1 μM LDN193189, 0.25 μM Sant1 and 100 nM Wnt-C59. At the end of Stage 3, the cells were dispersed by exposing to Accutase. The released cells were rinsed with DMEM-basic, and spun down at 300 g for 3 min. The cells were then seeded in 6-well AggreWell™ Microwell Plates (Stem Cell, Cat #27940) in Stage 4 medium supplemented with 10 μM Y27632, and spun down to the bottom of the microwells by centrifuging the plates at 300 g for 5 min. The cells were then incubated at 5% CO₂ at 37° C. for 20 h, and the generated cell clusters were transferred into ultra-low attachment 6-well plate (Beaverbio, Cat #40406) with Stage 4 medium. Suspended aggregates were cultured in an incubator shaker (Infors-HT, Multitron) at a rotation rate of 90 rpm, at 37° C., 5% CO₂, and 85% humidity.

Stage 4 (5-6 days). DMEM-basic supplemented with 1% Pen/Strep, 1% Glutamax, 1% B27, 100 ng/mL EGF, 0.2 μM TPB, 10 mM Nicotinamide, 0.25 μM Sant1 and 0.25 mM Vitamin C.

Stage 5 (6 days). DMEM-basic supplemented with 1% Pen/Strep, 1% Glutamax, 1% B27, 10 μM ALK5 inhibitor II, 0.3 μM LDN193189, 1 μM T3, 10 μM ISX9, 10 μg/mL heparin, 0.1 μM γ-secretase inhibitor Xxi, 100 nM Wnt-C59, 10 μM Y27632 and 0.25 mM Vitamin C.

Stage 6 (2-4 days). DMEM-basic supplied with 1% Pen/Strep, 1% B27, 10 μM ALK5 inhibitor II, 0.5 μM R428, 1 μM T3, 10 UM Forskolin, 10 μg/mL heparin, 10 μM zinc sulfate, 2 mM N-acetyl cysteine and 0.25 mM Vitamin C.

### Flow Cytometry

Differentiated cells were released into a single-cell suspension with Accutase, then stained for surface markers and intracellular marker. The antibodies used is listed in Table 2.

TABLE 2
Antibody information for flow cytometry.

| Antigen | Species | Source | Dilution | Cat# |
|---|---|---|---|---|
| PE anti-human | Mouse | BD Biosciences | 1:200 | 561589 |
| FOXA2 | | | | |
| APC anti-human | Mouse | BD Biosciences | 1:200 | 562594 |
| SOX17 | | | | |
| PDX1 | Goat | R&D | 1:200 | AF2419 |
| NKX6.1 | Mouse | DSHB | 1:200 | F55A12-c |
| C-Peptide | Rat | DSHB | 1:200 | GN-ID4 |
| GLUCAGON | Mouse | Sigma-Aldrich | 1:200 | G2654 |
| SOMATOSTATIN | Mouse | Santa Cruz | 1:200 | Sc-55565 |
| CHGA | Rabbit | ZSGB-Bio | 1:50 | ZA-0507 |

### Immunohistochemistry and Immunofluorescence Staining

Frozen tissue sections. Cell aggregates or tissue were washed with PBS and fixed with 4% PFA (Biosharp, Cat #BL539A) for 2 h (cell aggregates) or 24 h (tissue) at 4° C. Samples were washed three times with PBS and dehydrated overnight at 4° C. in 30% sucrose solution. The samples were overlaid with OCT (Sakura, Cat #4583) solution and frozen using liquid nitrogen and stored at −80° C. A freezing microtome was used to cut 10 μm sections, which were placed on slides. The slides were washed with PBS and permeabilized with PBST solution (PBS+0.2% Triton X−100+5% donkey serum) for 1 h at room temperature. Slides were incubated with primary antibodies diluted in PBST solution at 4° C. overnight. Following three washes with PBS, the slides were incubated with secondary antibodies conjugated to Alexa Fluor® 488, 555 or 647 (Life Technologies) in PBST solution at 1:1000 for 1 h and stained with DAPI for 5 min at room temperature. Images were captured using Leica TCS SP8 confocal microscope.

All antibodies used above are listed in Table 3.

TABLE 3
Antibody information for immunohistochemistry
and immunofluorescence staining.

| Antigen | Species | Source | Dilution | Cat# |
|---|---|---|---|---|
| PDX1 | Goat | R&D | 1:200 | AF2419 |
| NKX6.1 | Mouse | DSHB | 1:200 | F55A12-c |
| NKX6.1 | Rabbit | Novus | 1:200 | NBP1-49672 |
| NKX2.2 | Mouse | DSHB | 1:200 | 74.5A5-c |
| C-Peptide | Rat | DSHB | 1:200 | GN-ID4 |
| GLUCAGON | Mouse | Sigma-Aldrich | 1:200 | G2654 |
| GLUCAGON | Rabbit | Abcam | 1:500 | Ab92517 |
| MAFA | Rabbit | Novus | 1:200 | NB400-137 |
| SOMATOSTATIN | Mouse | Santa Cruz | 1:200 | Sc-55565 |
| CHGA | Rabbit | ZSGB-Bio | 1:50 | ZA-0507 |

qRT-PCR

Total RNA was extracted with RNeasy Mini Kit (QIAGEN, Cat #74004) following the manufacturer's instructions. Transcript One-Step GDNA removal and cDNA synthesis supermix (TransGen Biotech, Cat #AT311-03) was used to synthesize cDNA. KAPA SYBR® FAST Universal qPCR Mix (KAPA Biosystems, Cat #KK4601) was used for qRT-PCR analysis, which was performed on a BIO-RAD CFX384TM Real-Time System. All relative expression levels were normalized to the housekeeping gene GAPDH and the results were analyzed using the ΔΔCt method. The primers are listed in Table 4.

TABLE 4
Primer sequence

| | Forward | Reverse |
|---|---|---|
| | primer | primer |
| Gene | sequence | sequence |
| GAPDH | TGCACCACCA | GGCATGGACT |
| | ACTGCTTAGC | GTGGTCATGA |
| | | G |
| OCT4 | CCGAAAGAGA | ATGTGGCTGA |
| | AAGCGAACCA | TCTGCTGCAG |
| | G | T |
| NANOG | TTTGTGGGCC | AGGGCTGTCC |
| | TGAAGAAAAC | TGAATAAGCA |
| | T | G |
| PDX1 | CGGAACTTTC | AAGATGTGAA |
| | TATTTAGGAT | GGTCATACTG |
| | GTGG | GCTC |
| NKX6.1 | GGGCTCGTTT | CCACTTGGTC |
| | GGCCTATTCG | CGGCGGTTCT |
| | TT | |
| NKX2.2 | TTCCAGAACC | GGGCGTCACC |
| | ACCGCTACAA | TCCATACCT |
| | G | |
| MAFA | GCTCTGGAGT | CTTCAGCAAG |
| | TGGCACTTCT | GAGGAGGTCA |

### Glucose Stimulated Insulin Secretion (GSIS)

Krebs buffer was prepared as follows: 129 mM NaCl, 4.8 mM KCl, 2.5 mM CaCl₂), 1.2 mM MgSO₄, 1 mM Na₂HPO₄, 1.2 mM KH₂PO₄, 5 mM NaHCO₃, 10 mM HEPES, 0.2% BSA dissolved in deionized and sterile filtered water. Krebs buffer containing 2.8 mM glucose, 16.7 mM glucose, and 30 mM KCl were prepared and warmed to 37° C.

Static GSIS. hPSC-islets (20-50 clusters) or human islets (20-50 islets) were collected and placed in a 24-well plate, and then rinsed twice with Krebs buffer. Cells were incubated successively in Krebs buffer, Krebs buffer containing 2.8 mM glucose, Krebs buffer containing 16.7 mM glucose and Krebs buffer containing 30 mM KCl at 37° C. for 1 h. Supernatant was collected after each incubation and cells were rinsed with fresh Krebs buffer at each solution change. Supernatant samples were frozen at −80° C. until detection was conducted. After the assay, cells were dispersed into single cells with Accutase and counted with Countess™ II Automated Cell Counter.

Dynamic GSIS. The dynamic function of hPSC-islets was assessed with an automated perifusion system (Biorep® Perifusion System; BioRep). hPSC-islets were assayed with effluent collected at a 100 μL/min flow rate every minute, exposed to glucose Krebs buffer and KCl Krebs buffer. A 2.8 mM glucose Krebs buffer was perfused for the first 60 min to equilibration. Then, solutions were switched as follows: 2.8 mM glucose Krebs buffer for 15 min, 16.7 mM glucose Krebs buffer for 30 min, 2.8 mM glucose Krebs buffer for 15 min and 30 mM KCl Krebs buffer for 15 min.

### ELISA

C-peptide, insulin and glucagon levels were detected using human C-peptide ELISA kit (ALPCO, Cat #80-CPTHU-E10), human insulin ELISA kit (ALPCO, Cat #80-INSHUU-E10) and human glucagon ELISA kit (Mercodia, Cat #10-1271-01) according to the manufacturer's instructions.

### Electron Microscopy

hPSC-islets were processed by the Center of Cryo-Electron (CCEM), Zhejiang University. Grids were examined with a Tecnai G2 Spirit electron microscope.

### Transplantation in Mouse

All mouse experimental procedures were performed according to the Animal Protection Guidelines of Peking University, China. Six to eight week-old male CB17.Cg-PrkdcscidLystbg-J/Crl (Scid/Beige) mouse were purchased from Beijing Vital River Laboratory Animal Technology Co, Ltd..

Transplantation into STZ-treated diabetic mice. Diabetes was induced by intraperitoneal injection of 70 mg/kg STZ (Selleck, Cat #S1312) after 16 h fasting for 5 consecutive days. Approximately 3×10⁶ hPSC-islets cells were transplanted under the left kidney capsule. Fasting blood glucose levels after 16 h fasting were monitored weekly with a handheld glucometer (ROCHE, Cat #06870279001) using a tail bleed. Body weights of animals were measured weekly. Glucose-stimulated human C-peptide secretion was assessed by collecting blood sample after 16 h fasting and 30 min following glucose injection (2 g/kg, 30% solution, i.p.). For glucose tolerance tests, intraperitoneal injection of glucose (2 g/kg, 30% solution) was performed after 16 h fasting, and blood glucose levels were monitored at the predetermined time points (0 min, 5 min, 15 min, 30 min, 60 min, 90 min and 120 min). Plasma was frozen at −80° C. until human C-peptide analysis.

Transplantation into non-STZ-treated healthy mice. Approximately 3×10⁶ hPSC-islet cells were transplanted under the left kidney capsule. Blood sample after a 16 h fast was collected biweekly for fasting human C-peptide measurement. Plasma was frozen at −80° C. until human C-peptide analysis.

### Transplantation in Nonhuman Primate

All monkey experimental procedures were approved by the Institutional Animal Care and Use Committee of Institute of Medical Biology, Chinese Academy of Medical Science (Ethics number: DWLL201908013). Four male rhesus macaques (4 years of age, 4-5.5 kg) from the Institute of Medical Biology Chinese Academy of Medical Science were used as recipients for hCiPCs-islet transplantation.

Diabetes induction. Diabetes was induced according to a previously reported method. Briefly, a single dose of STZ (90 mg/kg, Adooq, Cat #A10868) was injected intravenously (within 5 min) after overnight fasting. STZ was diluted in 0.1 M citrate buffer (pH 4.3-4.5) and immediately administered rapidly intravenously followed by administration of normal saline (40-50 mL) for hydration. Omeprazole (0.5 mg/kg, Losec®, Astrazeneca AB) was injected to prevent nausea and vomiting after hydration. Blood glucose was monitored every hour over the first 12 h after STZ injection, and thereafter, 4 times a day. Exogenous insulin injections commenced 3 days after STZ treatment. The short-acting form of insulin (Humalog®, Eli Lilly Italia S.p.A.) and long-acting form of insulin (Lantus®, Sanofi-Aventis Deutschland GmbH) were injected subcutaneously. The levels of blood glucose, C-peptide, and HbA1c were recorded before hPSC-islet transplantation.

Transplant surgeries. The transplantation procedures were performed after the diabetic status of recipient monkey was confirmed. After the i.v. administration of Propofol (0.5 mL/kg, Petsun Therapeutics), monkeys were anaesthetized with inhalable isoflurane. Heart rate, temperature, blood oxygenation, and blood pressure were monitored in real-time during the surgical procedure. 5% glucose was infused to maintain blood glucose levels. A total of about 4-6×10⁸ hPSC-islet cells were infused into the portal vein through a jejunal vein after laparotomy.

To mitigate the instant thrombosis, low molecular weight heparin sodium (150 IU/kg, i.h., Fluxum®, Alfasigma S.p.A.) was injected subcutaneously at 2 h and 8 h postoperatively, followed by three times a day for about 1 week. Antibiotic treatment was continued for 7 days. Pain-relief medication was administered for first 3 days post cell infusion.

Routine tests. C-peptide secretion, body weight, HbA1c, complete blood count, serum creatinine and liver function analysis were routinely performed. The complete blood cell count was done using Sysmex XT-200i. HbA1c, serum creatinine and liver function analysis were assessed using Mindray BS-2000.

### Statistical Analysis

Data analysis was performed by using GraphPad Prism software. Statistical significance was evaluated by t-test. Throughout the manuscript, n represents number of biological replicates unless otherwise stated. P-values presented as follows: * P<0.05; ** P<0.005; *** P<0.0005; **** P<0.00005.

### Example 1. Characterization of hPSC-Islets

The hPSC-derived pancreatic islets (hPSC-islets) obtained by the method as described above was characterized in vitro.

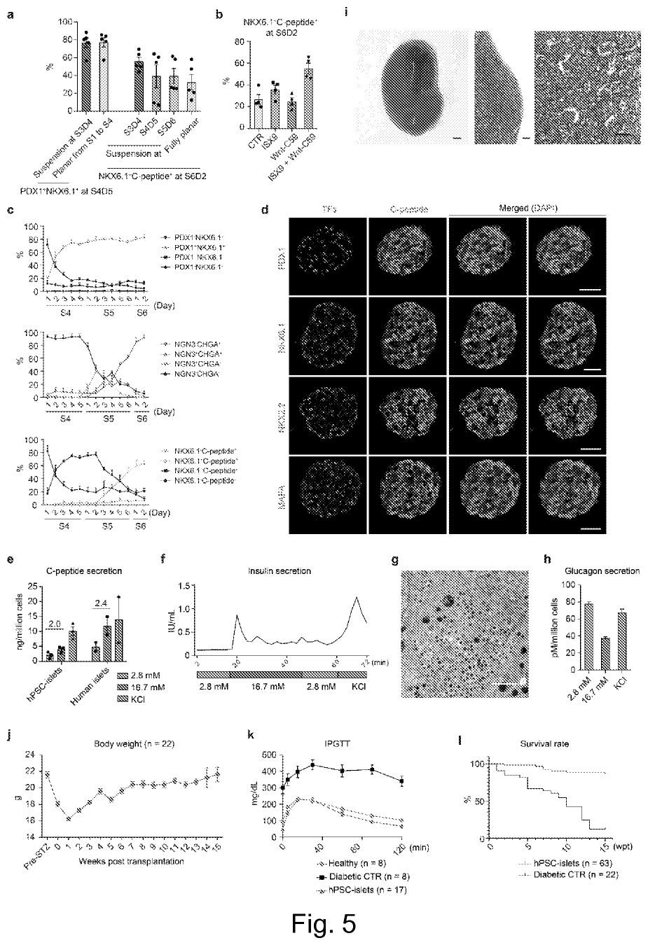

Immunostaining showed that most C-peptide positive cells co-expressed transcription factors of pancreatic endocrine and mature β cells (FIG. 5d). The levels of secreted C-peptide were comparable in hPSC-islets and primary human islets (FIG. 5e). Importantly, the ability to respond to glucose challenge with dynamic biphasic insulin secretion and the presence of dense-core, crystallized insulin granules suggested the mature insulin secretory function of hPSC-islets (FIGS. 5f and g).

In addition to β cells, glucagon (GCG) positive α cells and somatostatin (SST) positive δ cells were also identified in the aggregates (FIG. 1c). GCG secretion was detectable and was suppressed upon glucose challenge (FIG. 5h). Flow cytometry analysis revealed that the hPSC-islets contained approximately 60% β cells, 11% α cells and 7% δ cells on average (FIG. 1d).

Further, the function of hPSC-islets was validated on a routinely used immunodeficient mouse model. After transplantation under the kidney capsule of streptozotocin (STZ)-induced diabetic mice, hPSC-islets survived with marked vascularization and preserved cellular complexity, shown by the presence of C-peptide⁺β cells, GCG⁺α cells and SST^{+δ cells} 16 weeks post transplantation (wpt) (FIG. 1e and FIG. 5i). Fasting blood glucose levels of transplanted mice were restored to physiological levels, accompanied by increase in body weights (FIG. 1f and FIG. 5j). Glucose tolerance tests showed glucose-responsive human C-peptide secretion, as well as rapid glucose clearance (FIG. 1g and FIG. 5k). Fasting human C-peptide secretion increased steadily from 2 to 12 wpt, after which it was maintained at around 1 ng/ml for up to 36 weeks in non-diabetic mice (FIG. 1h). Notably, the 15-week survival rate of hPSC-islet transplanted diabetic mice was over 85%, compared to less than 20% in the non-transplanted control group (FIG. 5l). These results indicated that hPSC-islets could reverse diabetes in mouse model.

### Example 2. Compatibility Across Cell Lines

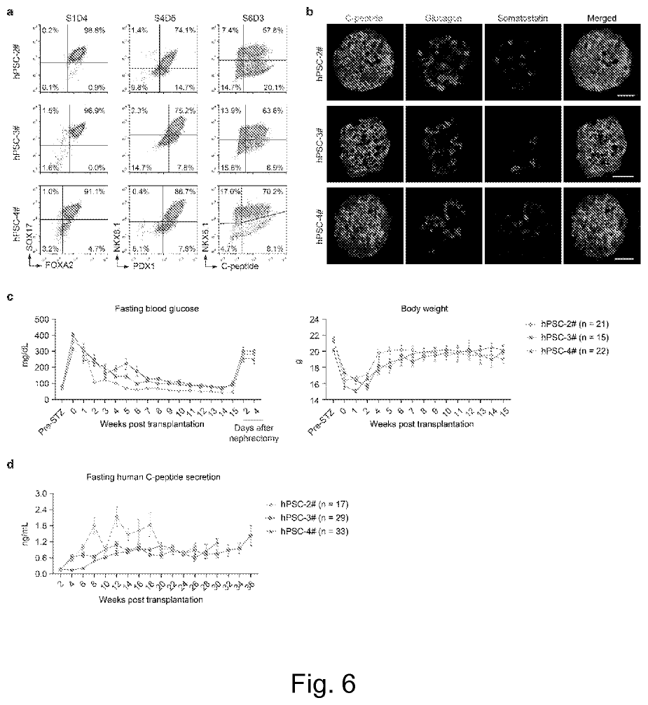

Furthermore, the established protocol demonstrated good compatibility across cell lines, shown by reproduction of similar in vitro characteristics and in vivo functionality on hPSC-islets derived from another 3 independent hPSC cell lines (FIG. 6 and Table 5). Notably, in all hPSC-islets transplanted mice, no tumorigenesis was observed (n=190).

TABLE 5
Flow cytometry data of multiple differentiation batches across cell
lines. Six representative independent batches are presented.

| | | Differentiation efficiency | | |
|---|---|---|---|---|
| | Batch | S1 | S4 | S6 |
| Cell line | number | (% of FOXA2⁺SOX17⁺) | (% of PDX1⁺NKX6.1⁺) | (% of NKX6.1⁺C-peptide⁺) |
| hPSC-#1 | 1 | 91.8 | 65.0 | 58.2 |
| | 2 | 93.0 | 75.2 | 66.0 |
| | 3 | 97.2 | 63.8 | 63.1 |
| | 4 | 99.0 | 75.0 | 64.8 |
| | 5 | 90.2 | 87.3 | 76.0 |
| | 6 | 93.1 | 75.5 | 60.0 |
| hPSC-#2 | 1 | 87.9 | 71.1 | 65.0 |
| | 2 | 71.8 | 82.0 | 71.0 |
| | 3 | 75.0 | 76.2 | 70.2 |
| | 4 | 99.0 | 75.7 | 57.1 |
| | 5 | 97.0 | 72.9 | 68.9 |
| | 6 | 98.6 | 85.7 | 62.8 |
| hPSC-#3 | 1 | 82.7 | 84.4 | 61.9 |
| | 2 | 90.0 | 90.3 | 60.2 |
| | 3 | 91.9 | 72.7 | 55.2 |
| | 4 | 94.2 | 85.2 | 60.1 |
| | 5 | 98.6 | 79.5 | 60.9 |
| | 6 | N/D | 62.0 | 58.0 |
| hPSC-#4 | 1 | 78.2 | 73.0 | 62.3 |
| | 2 | 96.0 | 92.0 | 67.0 |
| | 3 | 93.0 | 86.0 | 70.0 |
| | 4 | 90.0 | 85.0 | 68.0 |
| | 5 | 96.0 | 85.0 | 70.0 |
| | 6 | 88.3 | 83.7 | 68.0 |

N/D, not done.

### Example 3. Efficacy and Safety of hPSC-Islet Transplantation in Nonhuman Primate Model

Next, inventors investigated the efficacy and safety of hPSC-islet transplantation in a nonhuman primate model, which more closely mimics human as compared to mouse model.

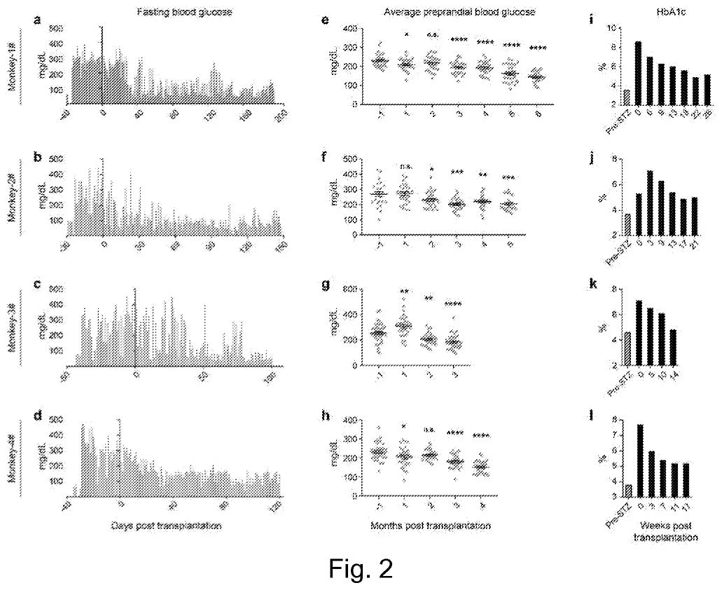

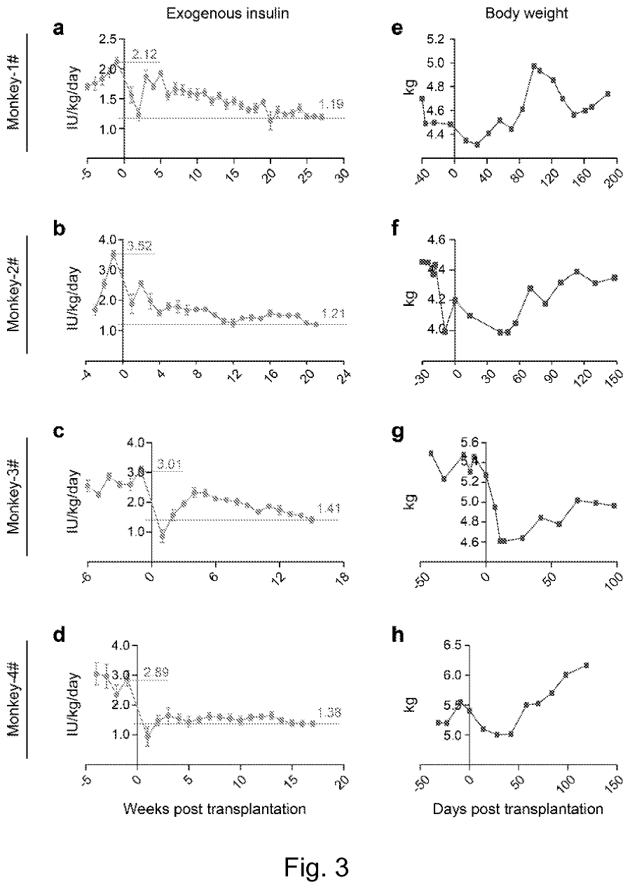

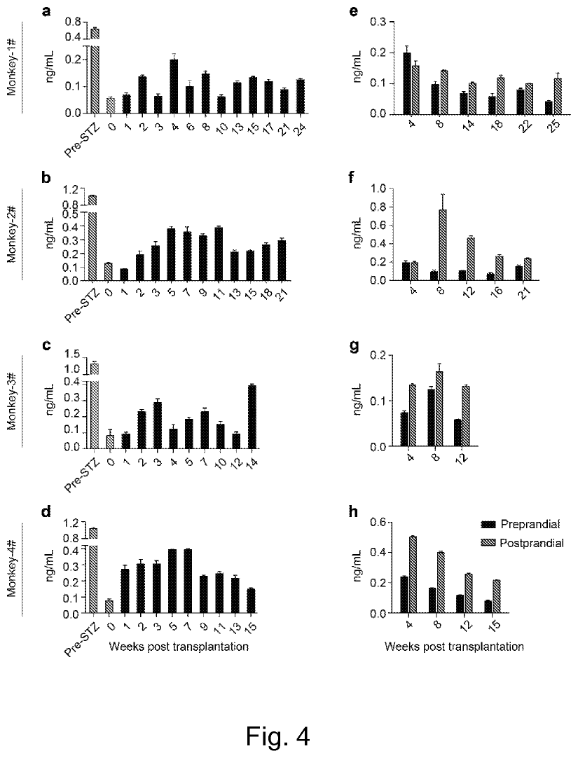

Four healthy adult rhesus macaques (Macaca mulatta) were used in this study (Table 6). All four macaques developed diabetes after a single high-dose STZ injection, resulting in fasting blood glucose levels over 200 mg/dL and C-peptide levels lower than 0.15 ng/ml (0.09±0.03 ng/ml) (FIG. 2a-d and FIG. 4a-d). Exogenous insulin was administered 3 days after the STZ injection. The exogenous insulin requirement of the four diabetic recipients ranged from 2 to 4 IU/kg per day (2.89±0.58 IU/kg/day) 1 week before cell transplantation, comparable to previous reports (FIG. 3a-d). Although all macaques were treated with intensive insulin therapy, levels of glycated hemoglobin A1c (HbA1c) dramatically increased from 3.9±0.5% to 7.2±1.4% within 1 to 2 months after STZ injection, indicating a rapid progression of diabetes (FIG. 2i-l). Notably, inventors observed that Monkey- #3 exhibited characteristics of labile diabetes, shown by swings in blood glucose levels that ranged from 40 to 545 mg/dL within a day and also fasting blood glucose levels that ranged from 40 to 450 mg/dL before cell transplantation (FIG. 2c).

To produce a ready-to-use cell source for macaque transplantation, inventors optimized a cryopreservation and recovery protocol for hPSC-islets. After generation, hPSC-islets were cryopreserved at single cell, and then recovered and reaggregated two days before infusion. The average viability and yield of hPSC-islets post recovery were 86.9%±1.6% and 82.0%±9.5% respectively (Table 6).

TABLE 6
Characterizations of transplanted hPSC-islets and their
respective diabetic rhesus macaque recipients.
Recipient

| | | | | Blood glucose of first |
|---|---|---|---|---|
| | | | Body | 2 days after STZ |
| | | | Weight | injection (before exogenous |
| | | Age | (kg) | insulin injection) |
| ID | Gender | (years) | (0 dpt) | (mg/dL) |
| #1 | M | 4 | 4.2 | 375; 212 |
| #2 | M | 4 | 4.9 | 231; 315 |
| #3 | M | 4 | 5.3 | 329; 334 |
| 4 | M | 4 | 5.4 | 453; 476 |

hCiPSC-islets

| | | | | | Flow cytometry | | | | | | |
|---|---|---|---|---|---|---|---|---|---|---|---|
| | | Transplanted | Recovery | | NKX6.1⁺C- | | | | | | |
| Recipient | | cell number | Viability | Yield | peptide⁺ | GCG⁺ | SST⁺ | qRT-PCR (Relative to S0) | | | |
| ID | Cell line | (*10⁸) | (%) | (%) | (%) | (%) | (%) | 4-Oct | PDX1 | NKX6.1 | NKX2.2 |
| #1 | hPSC-#2 | 4.7 | 87.8 | 94.8 | 70.9 | 7.9 | 7.9 | 6.29E−03 | 6.14E+02 | 1.24E+03 | 5.67E+02 |
| #2 | hPSC-#1 | 3.9 | 85.1 | 71.7 | 63.3 | 11 | 6.7 | 9.05E−03 | 8.13E+02 | 1.81E+03 | 9.61E+02 |
| #3 | hPSC-#3 | 5.3 | 88.7 | 80.7 | 62.9 | 12.8 | 6 | 1.08E−02 | 1.11E+03 | 2.03E+03 | 9.30E+02 |
| #4 | hPSC-#4 | 5.5 | 86.2 | 80.7 | 66.5 | 15.4 | 10 | 5.62E−03 | 1.30E+03 | 2.06E+03 | 9.82E+02 |

Recovered hPSC-islets were transplanted into the diabetic macaques at a single dose by intraportal infusion. After hPSC-islet transplantation, all four recipients exhibited relief from diabetic symptoms (FIGS. 2 and 3). Firstly, fasting blood glucose levels decreased and stabilized over time (FIG. 2a-d), especially in Monkey- #4, in whom an obvious downward trend was observed in the first month post transplantation (FIG. 2d). Secondly, the average preprandial blood glucose levels were also significantly decreased in all recipients after hPSC-islet infusion (FIG. 2e-h). Accordingly, HbA1c, a universal clinical measurement for glycemic control in diabetic patients, decreased from 7.20±1.4% before transplantation to 5.0±0.2% on average by the date of submission (FIG. 2i-l). Notably, these improvements were also seen in Monkey- #3, the recipient exhibiting a labile diabetes-like state post-STZ injection (FIG. 2c, g and k). Collectively, these results revealed that transplantation of hPSC-islets effectively lowered hyperglycemia and improved overall glycemic control in all diabetic macaques.

Furthermore, the exogenous insulin requirement dramatically decreased after hPSC-islet transplantation (FIG. 3a-d). At 1 to 2 weeks post hPSC-islet infusion, a dip in exogenous insulin requirement was seen in all recipients, which was likely due to decreased appetite in the recovery period following surgery and intense immunosuppression around the time of transplantation. Exogenous insulin requirement increased upon recovery to a normal diet. As hPSC-islets engrafted and matured in vivo, exogenous insulin requirement gradually decreased and stabilized over time. At 15 weeks post hCiPSC-islets infusion, exogenous insulin requirement in the four recipients decreased by 31% (from 2.12 to 1.46 IU/kg per day), 60% (from 3.52 to 1.41 IU/kg per day), 54% (from 3.09 to 1.41 IU/kg per day) and 52% (from 2.89 to 1.40 IU/kg per day) respectively, compared to pre-transplant levels (FIG. 3a-d). Meanwhile, body weights of recipient macaques increased after 6 weeks post hPSC-islet infusion (FIG. 3e-h).

C-peptide secretion was continuously monitored in all macaques. It has been observed a gradual increase of secreted C-peptide levels in all recipients within the first 1-to-2-month post hPSC-islet infusion, suggesting a functional maturation of hPSC-islets in vivo (FIG. 4a-d). Furthermore, C-peptide secretion responded to meal challenge starting from 4 to 8 wpt in all recipients (FIG. 4e-h). In Monkey- #2, the fold changes in postprandial C-peptide levels from fasting levels exceeded 3 from 8 to 16 wpt (FIG. 4f). The level of C-peptide secretion peaked within 8 wpt, and the average secretion level was 0.37±0.29 ng/mL at 8 wpt, a marked increase from pre-transplantation levels (0.09±0.03 ng/ml) (FIG. 4e-h). The significant increase of secreted C-peptide levels in all recipients was consistent with the observed improvement in glycemic control and decreased exogenous insulin requirements.

In summary, inventors demonstrate that islets derived from human pluripotent stem cells are able to survive, relieve hyperglycemia and improve overall glycemic control in the long term in a preclinical context. Firstly, transplantation of hPSC-islets effectively decreased HbA1c and restored endogenous C-peptide secretion (FIG. 2-4), positive outcomes that indicate control of disease progression. Clinical studies have associated every percentage point reduction of HbA1c with significant reduction in risk of diabetic-related complications. Additionally, the restoration of endogenous C-peptide has also been credited as the main factor associated with overall clinical benefit in clinical islet transplantation. Secondly, inventors observed that one recipient macaque presenting the features of labile diabetes benefited from hPSC-islet transplantation (FIG. 2c, g and k). Clinical reports showed strong evidence that refractory hypoglycemia, a predominantly life-threatening symptom in “brittle diabetes” patients, can be resolved with clinical islet cell transplantation. Although more animals should be tested, our data suggests the potential of hPSC-islet infusion in improving glycemic control and correcting severe hypoglycemia in this selected group of labile diabetic patients. Finally, the ability to be efficiently cryopreserved makes hPSC-islets a consistently available, ready-to-use cell source, which is especially important for clinical application, and affords much needed flexibility in transplantation into humans. Collectively, as the first comprehensive report on the long-term assessment of hPSC-islets in a primate model of diabetes, the data obtained in this study could provide valuable insight for stem cell derived islets in clinical research for diabetes treatment.

The disclosure has been described above with reference to embodiments thereof. It should be understood that various modifications, alterations and additions can be made by those skilled in the art without departing from the spirits and scope of the disclosure. Therefore, the scope of the disclosure is not limited to the above 5 particular embodiments but only defined by the claims as attached.

The invention claimed is:

1. A method of in vitro generating functional hPSC-islets that contain C-peptide⁺ cells, glucagon⁺ cells and somatostatin⁺ cells, comprising:
(1) culturing the hPSCs in a sixth culture medium to obtain cells expressing markers characteristic of the definitive endoderm;
(2) culturing the cells obtained in step (1) in a fifth culture medium to obtain cells expressing markers characteristic of primitive gut tube cells;
(3) culturing the cells obtained in step (2) in a fourth culture medium to obtain cells expressing markers characteristic of posterior foregut cells;
(4) culturing the cells obtained in step (3) in a third culture medium to obtain cells expressing markers characteristic of pancreatic progenitors;
(5) culturing the cells obtained in step (4) in a second culture medium to obtain cells expressing markers characteristic of pancreatic endocrine progenitors;
(6) culturing the cells obtained in step (5) in a first culture medium to obtain cells expressing markers characteristic of functional hPSC-islets;
wherein the second culture medium is supplemented with ISX9 or Wnt-C59, preferably ISX9.

2. The method of claim 1, wherein the second culture medium is supplemented with ISX9 and Wnt-C59.

3. The method of claim 1 or claim 2, wherein the first culture medium comprises a basal medium supplemented with one or more of an ALK5 inhibitor, an Adenylyl cyclase activator, an Axl inhibitor, an IκB kinase inhibitor, T3 and ZnSO₄.

4. The method of any one of claims 1 to 3, wherein the second culture medium comprises a basal medium further supplemented with one or more of an inhibitor of ALK5, a BMP signaling inhibitor, a thyroid hormone and an inhibitor of NOTCH signaling.

5. The method of any one of claims 1 to 4, wherein the third culture medium comprises a basal medium supplemented with one or more of an epithelial growth factor, an activator of protein kinase C, an inhibitor of Sonic hedgehog signaling and a component of the vitamin B complex.

6. The method of any one of claims 1 to 5, wherein the fourth culture medium is supplemented with one or more of retinoic acid (RA), an inhibitor of Sonic hedgehog signaling, and an inhibitor of BMP signaling.

7. The method of claim 6, wherein the fourth culture medium is further supplemented with an inhibitor of Wnt signaling.

8. The method of claim 7, wherein the inhibitor of Wnt signaling is Wnt-C59.

9. The method of any one of claims 1 to 8, wherein the fifth culture medium comprises a basal medium supplemented with an activator of FGF signaling.

10. The method of claim 9, wherein the fifth culture medium is further supplemented with a TGF-beta/Smad inhibitor, and/or a Wnt inhibitor.

11. The method of claim 10, wherein the Wnt inhibitor is Wnt-C59.

12. The method of any one of claims 1 to 11, wherein the sixth culture medium comprises a basal medium supplemented with one or more of an activator of Activin receptor, a Wnt activator, a ROCK inhibitor and an PI3K inhibitor.

13. The method of any one of claims 1 to 12, wherein step (1) further comprises culturing in the seventh culture medium after culturing in the sixth culture medium and before step (2), wherein the seventh culture medium comprises a basal medium supplemented with Glucose, L-glutamine, B27, Activin A, and Vitamin C.

14. The method of any one of claims 1 to 13, wherein the human pluripotent stem cells are embryonic stem cells or induced pluripotent stem cells.

15. The method of any one of claims 1 to 14, the culture of one or more of steps (1) to (6) is suspension culture.

16. The method of claim 15, the culture of step (1) to step (3) is suspension culture.

17. A population of cells comprising functional hPSC-islets obtainable by the method of any one of claims 1 to 16.

18. A pharmaceutical composition comprising the population of cells of claim 17.

19. A method for treating a mammal having, or at risk of having, type I diabetes, type II diabetes, pre-diabetes or any combination thereof, the method comprising administering to the mammal the population of cells of claim 17 or the pharmaceutical composition of claim 18.

20. A kit for generating functional hPSC-islets that contain C-peptide⁺ cells, glucagon⁺ cells and somatostatin⁺ cells, comprising:
at least one of a first to a seventh culture medium defined in any one of claims 1 to 16.

21. Use of ISX9 and Wnt-C59 in inducing differentiation of pancreatic progenitors into pancreatic endocrine progenitors.
